SEQUENCE LISTING (4 sequences)

SEQ ID NO: 1 - protein, 10 aa, Homo sapiens
YNWNSFGLRF

SEQ ID NO: 2 - protein, 9 aa, Artificial Sequence
  TAK-448 peptide
  MISC_FEATURE (1)..(1): Y is a D amino acid
  MISC_FEATURE (2)..(2): W is a D amino acid
  MOD_RES (6)..(6): G is aza-Gly
  MOD_RES (8)..(8): R is Arg(Me)
  MOD_RES (8)..(8): METHYLATION
  MOD_RES (8)..(8): R is Arg(Me)
YWNTFGLRW

SEQ ID NO: 3 - protein, 9 aa, Artificial Sequence
  TAK-683 peptide
  MOD_RES (1)..(1): Arg(Me)
  MOD_RES (1)..(1): R is Arg(Me)
YWNTFGLRW

SEQ ID NO: 4 - protein, 10 aa, Artificial Sequence
  kisspeptin peptide analog
FRLGFSNWNY

(19) United States
(12) Patent Application Publication
(10) Pub. No.: US 2022/0313781 A1
(43) Pub. Date: Oct. 6, 2022

(54) Kisspeptins to Predict and Treat Delayed Puberty

(71) Applicants: The General Hospital Corporation, Boston, MA (US); Children`s Medical Center Corporation, Boston, MA (US)

(72) Inventors: Stephanie Beth Seminara, West Newton, MA (US); Yee-Ming Chan, Canton, MA (US); Margaret Flynn Lippincott, Somerville, MA (US)

(21) Appl. No.: 17/616,144

(22) PCT Filed: Jun. 8, 2020
(86) PCT No.: PCT/US2020/036662

§ 371 Date: Dec. 2, 2021

Related U.S. Application Data
(60) Provisional application No. 62/858,479, filed on Jun. 7, 2019.

(51) Int. Cl. A61K 38/17; A61K 31/485; G01N 33/74; A61P 5/02

(52) U.S. Cl. CPC A61K 38/1709; A61K 31/485; G01N 33/74; A61P 5/02; G01N 2333/59; G01N 2800/048

(57) ABSTRACT

Methods of diagnosing and treating pathologic hypogonadotropic hypogonadism and reproductive endocrine dysfunction using kisspeptin and kisspeptin analogs.

### CLAIM OF PRIORITY

This application claims the benefit of U.S. Provisional Application Ser. No. 62/858,479, filed on Jun. 7, 2019. The entire contents of the foregoing are incorporated herein by reference.

### FEDERALLY SPONSORED RESEARCH OR DEVELOPMENT

This invention was made with Government support under Grant No. HD043341 awarded by the National Institutes of Health and FD005712 awarded by the Food and Drug Administration. The Government has certain rights in the invention.

### TECHNICAL FIELD

Described herein are methods of diagnosing and treating delayed puberty using kisspeptin.

### BACKGROUND

Idiopathic Hypogonadotropic Hypogonadism (IHH) is a rare disorder due to absence of GnRH neurons or their regulatory neurocircuits in the hypothalamus. It results in deficient secretion of luteinizing hormone (LH) and follicle stimulating hormone (FSH) as well as failure to achieve normal reproductive function by age 18. When accompanied by anosmia, IHH is referred to as Kallmann Syndrome (KS). Untreated, patients remain sexually infantile.

Children presenting with delayed puberty pose a vexing challenge for clinicians because of difficulty in predicting which children will eventually progress through puberty and which children will not (1,2). Some causes of delayed puberty are readily recognizable, such as primary gonadal insufficiency, anatomic lesions of the hypothalamic/pituitary region, and functional hypogonadotropic hypogonadism (i.e., physiologic suppression of the reproductive endocrine axis by chronic illness, stress, or negative energy balance). However, these conditions only account for delayed puberty in 36-47% of girls and 11-29% of boys presenting for pediatric endocrinology care (3-5). For the majority of girls and boys with delayed puberty, the provider is left with two potential diagnoses with markedly different outcomes: constitutional delay and IHH (1). Constitutional delay is a self-limited condition in which puberty starts late (or starts then stalls temporarily) but eventually begins and progresses to attainment of full adult reproductive endocrine function (6). In contrast, IHH is a pathologic disorder that requires treatment (7). Currently, there is no reliable method to prospectively distinguish constitutional delay from IHH.

### SUMMARY

Provided herein are methods of treating a subject who has a reproductive endocrine dysfunction, optionally pathologic hypogonadotropic hypogonadotropism. The methods include administering a therapeutically effective amount of (i) a plurality of doses (e.g., at least 2, 3, 4, 5, 6, 7, 8, 9, 10 or more, e.g., 2, 3, 4, 5, or 6/day) of kisspeptin or a kisspeptin analog and/or (ii) an opioid antagonist or mixed agonist-antagonist to the subject. Also provided herein are compositions comprising (i) a kisspeptin or a kisspeptin analog and/or (ii) an opioid antagonist or mixed agonist-antagonist for use in a method of treating a subject who has pathologic hypogonadotropic hypogonadotropism, the method comprising administering a plurality of doses of kisspeptin or a kisspeptin analog and/or (ii) an opioid antagonist or mixed agonist-antagonist.

In some embodiments, the plurality of doses are administered at 1-6 hour intervals, preferably at 2-3 hour intervals, over at least 2-3 days and preferably over at least 1, 2, 3, 4, 6, or 12 months.

In some embodiments, each of the plurality of doses comprises a dose equivalent to 0.08-2.4 nmol/kg kisspeptin-10, administered via intravenous bolus (WB), subcutaneous injection (SC), or via a pump.

In some embodiments, each of the plurality of doses comprises a dose equivalent to 0.2-0.3 nmol/kg kisspeptin-10, preferably 0.24 nmol/kg kisspeptin-10.

In some embodiments, the plurality of doses comprises at least one supraphysiologic dose equivalent to 2.4-24 nmol/kg kisspeptin-10. In some embodiments, the at least one supraphysiologic dose is administered as a first dose, or first two or more doses, optionally all, of the plurality of doses.

In some embodiments, the methods include administering a therapeutically effective amount of an opioid antagonist or mixed agonist-antagonist to the subject. In some embodiments, the opioid antagonist is naloxone or naltrexone, or the mixed agonist-antagonist is buprenorphine.

In some embodiments, the methods further include administration of one or more gonadotropins (e.g., luteinizing hormone (LH) and/or follicle-stimulating hormone, (FSH)).

Also provided herein are methods for identifying a subject as having, being at risk for, or having an attenuated form of, a reproductive endocrine dysfunction (RED), e.g., pathologic hypogonadotropic hypogonadotropism. The methods include measuring a baseline level of LH in the subject; administering a stimulating dose of kisspeptin or a kisspeptin analog to the subject, e.g., a dose comprising to 0.08-15 nmol/kg kisspeptin-10, administered via intravenous bolus (WB) or a dose comprising 0.8-500 nmol/kg kisspeptin-10, administered via subcutaneous (SC) injection; measuring at least one level of LH after administration of the stimulating dose, e.g., within about 10, 15, 20, 30, 45, or 60 minutes after administration of the stimulating dose; comparing the baseline level of LH in the subject to the level of LH after administration of the stimulating dose, and identifying a subject with delayed puberty who has a level of LH after administration of the stimulating dose that is similar to the baseline level of LH (e.g., less than 3.5, 3, 2.5, or 2 times the baseline level) as having a RED, e.g., pathologic hypogonadotropic hypogonadotropism.

Alternatively, the methods can include administering a stimulating dose of kisspeptin or a kisspeptin analog to the subject, e.g., a dose comprising to 0.08-15 nmol/kg kisspeptin-10, administered via intravenous bolus (WB) or a dose comprising 0.8-500 nmol/kg kisspeptin-10, administered via subcutaneous (SC) injection; measuring at least one level of LH after administration of the stimulating dose, e.g., within about 10, 15, 20, 30, 45, or 60 minutes after administration of the stimulating dose; comparing the level of LH after administration of the stimulating dose to a reference level, and identifying a subject with delayed puberty who has a level of LH after administration of the stimulating dose that is below (or not significantly different from) the reference level of LH as having a RED, e.g., pathologic hypogonadotropic hypogonadotropism.

In some embodiments, the methods include administering a treatment for the reproductive endocrine dysfunction, e.g., pathologic hypogonadotropic hypogonadotropism, to the identified subject.

In some embodiments, the treatment includes administering a plurality of doses of kisspeptin or a kisspeptin analog. In some embodiments, the plurality of doses are administered at 1-6 hour intervals, preferably at 2-3 hour intervals, over at least 2-3 days and preferably over at least 1-12 months. In some embodiments, each of the plurality of doses comprises a dose equivalent to 0.08-2.4 nmol/kg kisspeptin-10, administered via intravenous bolus (IVB), subcutaneous injection (SC), or via a pump. In some embodiments, each of the plurality of doses comprises a dose equivalent to 0.2-0.3 nmol/kg kisspeptin-10, preferably 0.24 nmol/kg kisspeptin-10. In some embodiments, the plurality of doses comprises at least one supraphysiologic dose equivalent to 2.4-24 nmol/kg kisspeptin-10. In some embodiments, the at least one supraphysiologic dose is administered as a first dose, or first two or more doses, optionally all, of the plurality of doses.

In some embodiments, the methods include administering a therapeutically effective amount of an opioid antagonist or mixed agonist-antagonist to the subject. In some embodiments, the opioid antagonist is naloxone or naltrexone, or the mixed agonist-antagonist is buprenorphine.

In some embodiments, the treatment further comprises administering gonadal steroid replacement therapy, e.g., testosterone in males and estrogen and/or progesterone/progestin in females.

In some embodiments, the methods further include administration of one or more gonadotropins (e.g., luteinizing hormone (LH) and/or follicle-stimulating hormone, (FSH)).

In some embodiments, the plurality of doses are administered at 1-6 hour intervals, preferably at 2 hour intervals, over at least 2-3 days and preferably over at least 1-12 months.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Summary of recruitment and participation.

FIGS. 2A-C. Neuroendocrine characteristics of children presenting with delayed or stalled puberty. A schematic of the protocol is shown in panel A. Details of the protocol are given in ref. (20). At the first visit, participants had serum LH measured to assess spontaneous pulsatility overnight and to chart responses to kisspeptin and GnRH. Participants then received exogenous pulsatile GnRH to enhance pituitary responsiveness to GnRH. They then returned for a second visit to measure LH secretion in response to kisspeptin and GnRH after this pituitary “priming.” Results are shown for Participant A, a “kisspeptin nonresponder” (B), and for Participant B, a “kisspeptin responder” (C).

FIG. 3. Distinct responses to kisspeptin in children who progressed through puberty and those who did not. Girls (open circles) and boys (filled circles) presenting with delayed or stalled puberty underwent kisspeptin-stimulation testing to assess the change in luteinizing hormone in response to exogenous kisspeptin (ΔLHₖᵢₛₛₚₑₚₜᵢₙ). Participants were then followed until age 18 years to determine whether they progressed through puberty spontaneously.

FIGS. 4A-D. Additional hormonal evaluation of children who progressed or did not progress through puberty. Girls (open circles) and boys (filled circles) presenting with delayed and stalled puberty were evaluated for serum luteinizing hormone (LH) and follicle-stimulating hormone (FSH) at the time of presentation (A and B, respectively) and the change in LH in response to exogenous gonadotropin-releasing hormone (GnRH, panel C). Boys were additionally evaluated for serum inhibin B (D).

FIGS. 5A-D: LH response to kisspeptin. Dotted lines represent time points of administration. (▾)=LH pulses determined by modified Santen & Bardin criteria. A: Healthy male. Genetics not assessed. Kisspeptin 0.313 ug/kg WB at 6 h. (+) response. B: KS male. PROKR2 c.518T>G p.L173R het; DMXL2 c.4016T>G p.V1339G het. Kisspeptin 0.313 ug/kg IVB at 6 h. (−) response. C. KS female. Genetics not assessed. LH responses graphed corresponding to kisspeptin dose. (+) LH response across range of doses. D. KS male. CHD7 c.2417T>C p.M806T het; PROKR2 c.254G>A p.R85H het. Same dosing as in panel C. (+) LH response. All data is graphed on the same Y axis.

FIG. 6: Data from FIG. 5C re-graphed, showing KS female response to kisspeptin at variable doses at a 2 h frequency

FIGS. 7A-C: Baseline Neuroendocrine Profiling. A. Study schema; B. Study subjects with DM who underwent 8 hr sampling between 2010-2011; C. Healthy sister in early follicular phase (EFP). E2=estradiol, P=progesterone, LH=luteinizing hormone.

FIGS. 8A-B: Baseline Studies with Response to Kisspeptin and GnRH. A. Study schema; B. Study subjects. Arrows indicated luteinizing hormones pulses detected by the algorithm. K=kisspeptin-10 by intravenous boluses and subscript indicates the dose 1=0.24 nmol/kg, 2=0.72 nmol/kg, 3=2.4 nmol/kg. G=GnRH WB 75 ng/kg. E2=estradiol, FSH=follicle stimulating hormone, LH=luteinizing hormone.

FIGS. 9A-B: Response to Kisspeptin Infusion and GnRH. A. Study schema; B. Study subject. Arrows indicated luteinizing hormones pulses detected by the algorithm. G=GnRH WB 75 ng/kg. E2=estradiol, FSH=follicle stimulating hormone, LH=luteinizing hormone.

FIGS. 10A-B: Neuropeptide Administration with Response to Kisspeptin and GnRH. A. Study schema; B. Study subjects. Arrows indicated luteinizing hormones pulses detected by the algorithm. K=kisspeptin-10 by intravenous boluses and subscript indicates the dose 1=0.24 nmol/kg, 2=0.72 nmol/kg, 3=2.4 nmol/kg. G=GnRH WB 75 ng/kg. E2=estradiol, FSH=follicle stimulating hormone, LH=luteinizing hormone.

FIG. 11: Kisspeptin administration to Tac2 Knock-out mice and littermate controls across sexual development. Dashed line=kisspeptin administration. Luteinizing hormone values are mean±SEM for each timepoint.

FIGS. 12A-F. LH pulse profile (A and D) and the effects of naloxone (NLX) (B, C, E, and F) in adult OVX WT and Tac2 Knock-out mice. A and D: LH pulses 120 min before NLX injection, and 180 min after NLX injection; NLX injection indicated by arrows. Arrowheads indicate the LH pulses. B and E: Changes in LH secretion (mean±SEM) 60 min before and 120 min after NLX in WT and OVX Tac2KO mice, respectively. C and F: The effects of NLX treatment on LH release are also shown as mean±SEM from 20 min before (Pre NLX) and 20 min after NLX injection (Post NLX). *P<0.05, Student t test.

### DETAILED DESCRIPTION

Despite nearly 50 years since the discovery of GnRH (31), understanding the factors that drive the onset of sexual maturation and subsequently maintain reproductive function remains a challenge that complicates diagnosis and treatment. Patients with idiopathic hypogonadotropic hypogonadism (IHH) are a key population to uncover these signals, as they have abnormal GnRH secretion/action (32, 33). Most IHH patients present as teens with delayed pubertal development and suffer life-long sexual infantilism and infertility if left untreated (32, 33).

Described herein are methods of diagnosing and treating delayed puberty using kisspeptin.

### Diagnosing Pathologic Hypogonadotropic Hypogonadotropism

For decades, clinicians and researchers have tried to develop tests to predict whether a child with pubertal delay will eventually progress through puberty (8). These tests include measurement of baseline (unstimulated) serum gonadotropins (luteinizing hormone, LH, and follicle-stimulating hormone, FSH), gonadotropins after stimulation with GnRH or GnRH analogs, and inhibin B at baseline. Unfortunately, all of these tests have significant deficits in sensitivity, specificity, or both (8). For example, studies have demonstrated significant overlap in serum inhibin B concentrations between boys with constitutional delay and those with IHH, with 10-29% of those with constitutional delay and 22-45% of those with IHH having an inhibin B that falls in the range of overlap (9-11). Despite an ever-growing understanding of the genetics of IHH and constitutional delay, genetic testing is currently inadequate for predicting pubertal outcomes. Currently, less than half of IHH patients have an identifiable mutation in an IHH gene (7). Furthermore, even if a mutation is found, the predictive power is limited by variable penetrance and expressivity; within the same family, some carriers of an IHH gene mutation may have IHH and others may have constitutional delay (24).

Children with delayed puberty who later progress through puberty are often indistinguishable at first presentation from those who have abiding hypogonadotropism, and years of waiting may be needed before a given child's outcome is known. As a result, patients, families, and providers can be faced with a conundrum when deciding between two common management approaches for delayed puberty: treatment with sex steroids or watchful waiting without intervention.

Kisspeptin is a neuropeptide secreted in the hypothalamus that potently stimulates GnRH secretion in all mammalian species studied to date, including humans (12). In studies by our group and others, reproductively intact adults responded to a single bolus of kisspeptin with a robust increase in LH, whereas adults with IHH largely did not (13-19). Thus, provocative testing using kisspeptin can be used to assess an individual's capacity for GnRH secretion.

Children with delayed or stalled puberty have a range of responses to kisspeptin, from no response to robust responses (20). Described herein are the results of a prospective study showing that a child's ability to respond to kisspeptin can predict whether that child will subsequently progress through puberty.

The kisspeptin-stimulation test described herein overcomes two fundamental challenges in predicting pubertal outcomes for children with delayed puberty. The first challenge is that there has not been a method to distinguish the physiologic hypogonadotropic hypogonadism of a normal prepubertal child from the pathologic hypogonadotropic hypogonadotropism of a child who has IHH. Being a provocative test, the kisspeptin-stimulation test provides the first method to measure a child's future potential for GnRH secretion. Indeed, we found that kisspeptin could elicit LH responses in children who eventually progressed through puberty at a time when they appeared prepubertal on physical examination and daytime laboratory evaluation.

The second challenge is distinguishing a child with a temporary pause in pubertal development from a child with IHH and partial pubertal development that has permanently stalled. We had previously shown that adults with IHH and partial reproductive endocrine activity fail to respond to kisspeptin (19). Similarly, in the current study, one participant who exhibited partial reproductive endocrine activity (Participants 8) had diminished responses to kisspeptin that correctly predicted his lack of pubertal development by 18 years, whereas inhibin B and GnRH-induced LH suggested that he would later progress through puberty.

As described in Example 1, in two participants (Participants B and 11) the lack of LH pulses on overnight measurements did not correctly anticipate the participants' eventual progress through puberty, whereas the kisspeptin-stimulation test accurately predicted the participants' outcomes. These participants had been receiving sex-steroid treatment at the time of their study visits, and this may have suppressed endogenous LH secretion. In contrast, their responses to kisspeptin were robust, and thus the kisspeptin-stimulation test reliably predicted their eventual pubertal progression even when performed in the context of exogenous sex-steroid treatment (e.g., testosterone in males and estrogen and/or progesterone/progestin in females).

Some patients with IHH may have intact responses to kisspeptin (e.g., those with mutations in TAC3 and TACR3, the genes for neurokinin B and its receptor, which appear to function upstream of kisspeptin, or in KISS1, which encodes kisspeptin itself) (25,26). However, this is likely to be only a small subset of DM patients, as mutations in TACR3 are present in only about 5% of patients with normosmic IHH, and mutations in TAC3 and KISS1 are rare causes of IHH (27-30).

Thus, the present methods can be used to diagnose pathologic hypogonadotropic hypogonadotropism, e.g., IHH, in subjects, e.g., mammalian, e.g., human subjects. In some embodiments the subjects are at least 10, 11, 12, 13, 14 or 15 years old, and/or are under the age of 18, 19, or 20 years, e.g., are 10-20 years old, 10-19, 10-18, 11-18, 11-19, 11-20, 12-18, 12-19, or 12-20 years old. The methods can also be used to detect dysfunction in reproductive endocrine function or partial or late-onset forms of pathologic hypogonadotropic hypogonadism, e.g., in subjects of any age. In some embodiments, the methods are used to diagnose congenital forms of pathologic hypogonadotropic hypogonadism, e.g., in children of any age, including neonates.

In some embodiments, although the current formal clinical definition of IHH requires a patient to be 18 years or older, in a subject who is less than 18 years old, the present methods determine that they have IHH, allowing this diagnosis to be made years earlier than the traditional age cutoff. In some embodiments, the present methods determine that they are at risk of developing IHH or have a high likelihood of developing IHH.

The methods rely on detection of levels of LH in response to administration of at least one stimulating dose of kisspeptin or a kisspeptin analog, e.g., a dose equivalent to at least 0.08, 0.1, 0.12, 0.14, 0.16, 0.18, 0.20, 0.22, or 0.24 nmol/kg kisspeptin-10, up to about 8, 10, 12, 14, or 15 nmol/kg kisspeptin-10, e.g., 0.08-15 nmol/kg kisspeptin-10, e.g., 0.1-12 nmol/kg kisspeptin-10, e.g., 0.2-10.11 nmol/kg kisspeptin-10, e.g., 0.2-0.5 nmol/kg kisspeptin-10, e.g., 0.22-0.25 nmol/kg kisspeptin-10, e.g., 0.24 nmol/kg kisspeptin-10, preferably administered as an intravenous bolus (WB). The dose can also be administered, e.g., as a subcutaneous (SC) or intranasal bolus. When administered SC, the dose is higher than the IV dose range, e.g., 10, 20 or about 30-times higher than the dose ranges above, e.g., at least 0.8 nmol/kg up to about 500, 600, or 750 nmol/kg. Intranasal doses are typically ˜10× subcutaneous doses, so 24 to 5000, 6000, or 7500 nmol/kg.

The methods can include obtaining at least one, e.g., one or more, sample from a subject, and evaluating the level of LH in the sample, and comparing the level with one or more references, e.g., a control reference that represents a normal level of LH, e.g., a level in a subject who does not have pathologic hypogonadotropic hypogonadotropism, and/or a disease reference that represents a level of LH in a subject having pathologic hypogonadotropic hypogonadotropism. Suitable reference values can include those shown in the Examples below. In preferred embodiments, the methods include obtaining a plurality of samples from the subject, e.g., before, during, and after administration of the stimulating dose, and determining levels of LH in the samples, and comparing the levels of LH in the samples before administration of the stimulating dose (e.g., baseline levels) to levels after administration of the stimulating dose. Thus the methods can include comparing the absolute LH level after kisspeptin administration to a reference (e.g., a reference representing a cohort of subjects, or baseline level in the subject); and/or determining a change in LH level from baseline to after stimulation, either as a) the unit increase (difference), b) the relative change (ratio). In some embodiments, subjects with LH ratios of 3.5. 4, 4.5 or greater, e.g., 4.85 or greater, are identified as having constitutional delay, while those who had LH ratios below 3.5, e.g., below 3, e.g., 2.33 or less are identified as having pathologic HH.

As used herein the term “sample”, when referring to the material to be tested for the presence of LH using a method described herein includes inter alia whole blood, plasma, serum, or urine.

Various methods are well known within the art for the quantification of LH from a sample. The methods can include isolation and/or purification of LH from the sample before quantification. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of LH can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art. Immunometric assays can be used.

Mass spectrometry, and particularly matrix-assisted laser desorption/ionization mass spectrometry (MALDI-MS) and surface-enhanced laser desorption/ionization mass spectrometry (SELDI-MS), are useful for the detection of LH. (See U.S. Pat. Nos. 5,118,937; 5,045,694; 5,719,060; 6,225,047).

Specific assays for LH are described in Wheeler, Methods Mol Biol. 2006; 324:109-24; Beastall et al., Ann Clin Biochem. 1987 May; 24 (Pt 3):246-62; Pappa et al., Theriogenology. 1999 Apr. 1; 51(5):911-26.

In some embodiments, the level of LH after kisspeptin stimulation is comparable to the presence and/or level of the protein(s) in the disease reference, and the subject has one or more symptoms associated with pathologic hypogonadotropic hypogonadotropism, then the subject has pathologic hypogonadotropic hypogonadotropism. In some embodiments, the subject has no overt signs or symptoms of pathologic hypogonadotropic hypogonadotropism, but the presence and/or level of one or more of the proteins evaluated is comparable to the presence and/or level of the protein(s) in the disease reference, then the subject has an attenuated form of pathologic hypogonadotropic hypogonadism and/or an increased risk of developing pathologic hypogonadotropic hypogonadotropism. In some embodiments, the subject has signs or symptoms of pathologic hypogonadotropic hypogonadotropism, but has a normal LH response, then the subject is sent for additional testing. In some embodiments, once it has been determined that a person has pathologic hypogonadotropic hypogonadotropism or has an increased risk of developing pathologic hypogonadotropic hypogonadotropism, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of LH, e.g., a control reference level that represents a normal level of LH, e.g., a level in an unaffected subject or a subject who is not at risk of developing a disease described herein, and/or a disease reference that represents a level of LH associated with pathologic hypogonadotropic hypogonadotropism, e.g., a level in a subject having DM or KS.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point (e.g., before stimulation with kisspeptin or a kisspeptin analog). Thus in some embodiments the methods can include calculating a ratio of levels or difference in levels, and detecting the presence of a change in the level of LH after stimulation with kisspeptin or a kisspeptin analog.

Subjects associated with predetermined values are typically referred to as reference subjects. For example, in some embodiments, a control reference subject does not have a disorder described herein (e.g. pathologic hypogonadotropic hypogonadotropism). In some cases it may be desirable that the control subject is male and in other cases it may be desirable that a control subject is female, and a reference level established that is used for a subject of the same sex. In some cases it may be desirable that the control subject has pathologic hypogonadotropic hypogonadotropism, and in other cases it may be desirable that a control subject does not have pathologic hypogonadotropic hypogonadotropism (e.g., is a subject who has constitutional delayed puberty but will eventually undergo puberty on their own without intervention).

A disease reference subject is one who has pathologic hypogonadotropic hypogonadotropism.

Thus, in some cases the level of LH in a subject being less than or equal to a reference level of LH (or change in LH after stimulation) is indicative of a clinical status (e.g., indicative of pathologic hypogonadotropic hypogonadotropism). In other cases the level of LH in a subject being greater than or equal to the reference level of LH is indicative of the absence of disease or normal risk of the disease. In some embodiments, the amount by which the level in the subject is the less than the reference level is sufficient to distinguish a subject from a control subject, and optionally is statistically significantly less than the level in a control subject. In cases where the level of LH in a subject being equal to the reference level of LH, the “being equal” refers to being approximately equal (e.g., not statistically different).

In a prepubertal child with gonadal insufficiency (as opposed to insufficiency at the level of the brain or pituitary gland, i.e., hypogonadotropic hypogonadism), the response to kisspeptin may be larger than in general population. They would still have “normal risk” of pathologic hypogonadotropic hypogonadism.

The predetermined value can depend upon the particular population of subjects (e.g., human subjects) selected. For example, an apparently healthy population will have a different ‘normal’ range of levels of LH than will a population of subjects which have, are likely to have, or are at greater risk to have, pathologic hypogonadotropic hypogonadotropism. Accordingly, the predetermined values selected may take into account the category (e.g., sex, age, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

### Methods of Treatment

The present methods can include treatments for pathologic hypogonadotropic hypogonadotropism. In some embodiments, the treatment is administered to a subject who is identified by a method described herein.

The methods described herein include methods for the treatment of disorders associated with pathologic hypogonadotropic hypogonadotropism. In some embodiments, the disorder is DM or KS (e.g., if the subject has anosmia). Generally, the methods include administering a therapeutically effective amount of kisspeptin or a kisspeptin analog as described herein, to a subject who is in need of, or who has been determined to be in need of, such treatment. The methods can also be used to treat subjects who have hypothalamic amenorrhea, e.g., due to states of negative energy balance (e.g., undernourished, anorexic, or athletes), hyperprolactinemia, adult-onset hypogonadotropic hypogonadism, medication effects (e.g., from glucocorticoids, opioids) or attenuated forms of pathologic hypogonadotropic hypogonadism and/or those at risk for developing pathologic HH, e.g., subjects with idiopathic infertility, irregular menstrual cycles, or abnormal semen analysis.

As used in this context, to “treat” means to ameliorate at least one symptom of the disorder associated with pathologic hypogonadotropic hypogonadotropism. Often, pathologic hypogonadotropic hypogonadotropism results in absence of puberty, e.g., by 18 years of age; poorly developed or undeveloped secondary sexual characteristics, or infertility; thus, a treatment can result in onset of puberty, development of secondary sexual characteristics and/or a return of fertility. Administration of a therapeutically effective amount of a compound described herein for the treatment of pathologic hypogonadotropic hypogonadotropism will result in one or more of increased levels of LH, increased levels of sex steroids (e.g., testosterone in males and estrogen and/or progesterone/progestin in females), and gametogenesis, e.g., in some cases increased levels of LH in response to administration of a stimulating dose of kisspeptin or a kisspeptin analog.

Thus, in some embodiments provided herein are methods for treatment of pathologic hypogonadotropic hypogonadotropism, e.g., DM or KS. The methods can comprise administration of one or more doses, e.g., supraphysiologic and/or physiologic or near-phsyiologic doses, of kisspeptin or a kisspeptin analog. In some embodiments, the doses can be administered, e.g., periodically, e.g., every 1-6, 1-4, 2-6 or 2-4 hours over a period of days, weeks, months, or years, e.g., for 2-4 days, e.g., for 48 hours, optionally repeated once a month, once every other month, once every three months, once every four months, once every 6 months, once every 8 months, once every 10 months, or once a year, optionally administered chronically, e.g., every day for a period of days, weeks, months, or years. The treatment can be continued until, and optionally stopped or reduced when, a desired outcome, e.g., fertility, or development of secondary sexual characteristics, has been achieved. In some embodiments, at least some of the doses are at physiological or near-physiological levels, e.g., 0.08-2.4, e.g., 0.1-5, 0.2-0.4, e.g., 0.24 nmol/kg IVB. In some embodiments, the methods include administering at least one dose that is supraphysiological, e.g., equivalent to 2-25, e.g., 2.4-24 nmol/kg kisspeptin-10, e.g., at least 2, 2.4, 2.5, 2.6, 3, 4, 5, 6, 7, 8, 9, 10, 11, 12, 13, 14, 15, 16, 17, 18, 19, or 20 nmol/kg kisspeptin-10, up to 10, 11, 12, 13, 14, 15, 16, 17, 18, 19, 20, 21, 22, 23, 24, or 25 nmol/kg kisspeptin-10. In some embodiments, the doses are administered SC, with physiologic doses of 0.8 to 60 nmol/kg, and supraphysiologic doses of 60 to 1200 nmol/kg.

In some embodiments, the administration is, e.g., intravenous, subcutaneous, or nasal. In some embodiments, the methods include using a pump to administer the dose. Thus administration can take place by means of an infusion pump, e.g., in the form of a device or system borne by a subject or patient and comprising a reservoir containing a liquid composition comprising an active agent and an infusion pump for delivery/administration of the composition to the subject or patient, or in the form of a corresponding miniaturized device suitable for implantation within the body of the subject or patient, e.g., a device similar to an insulin pump, that can deliver periodic boluses of the active agents.

In some embodiments, the methods include repeating a kisspeptin-stimulation test as described above, e.g., after 2-3 days. In some embodiments, the treatment continues for months and years until all desired effects. For example, for ovulation induction in women, a course could be anywhere from 1 month to 6 months. For spermatogenesis induction in men, a course could be anywhere from 3 to 24 months. For pubertal induction and/or maintenance of reproductive endocrine function, treatment could be for years.

The methods can also or alternatively include administration of an opioid antagonist, and/or a conventional treatment for pathologic hypogonadotropic hypogonadotropism.

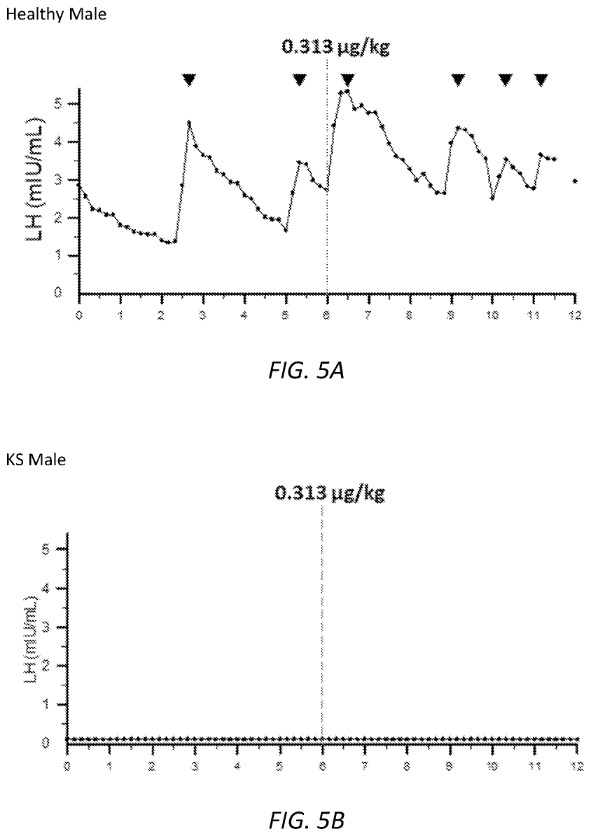

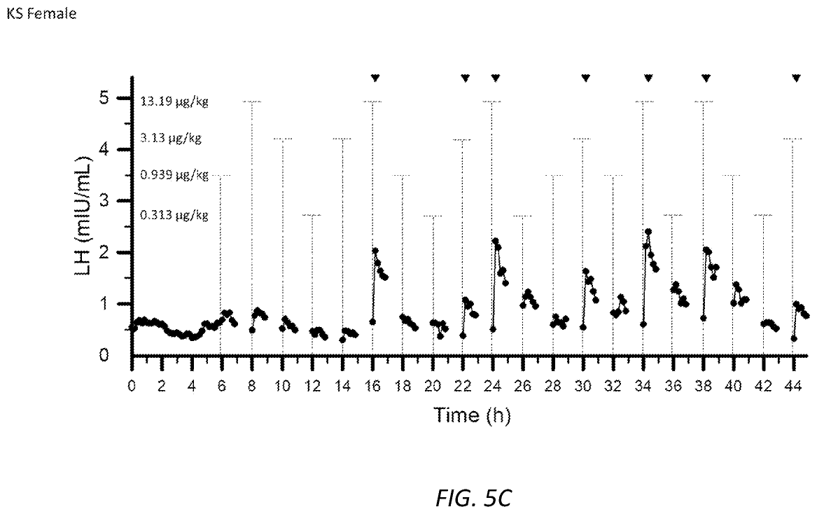

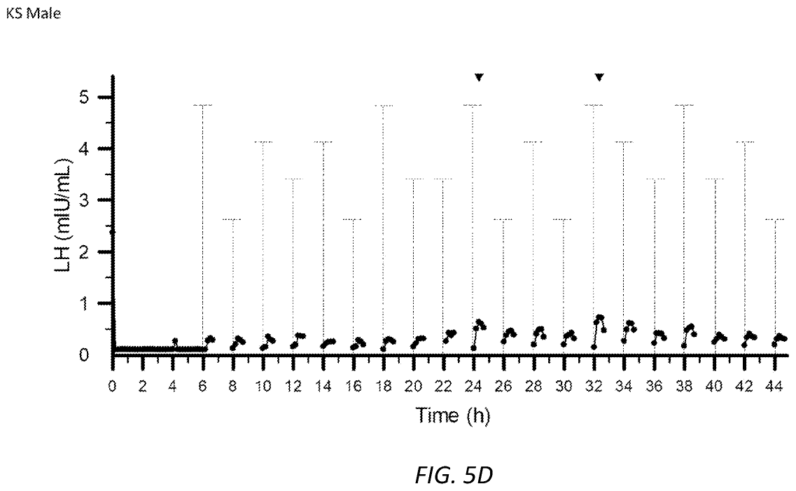

Kisspeptin resistance can occur in normosmic and anosmic forms of hypogonadotropism, as FIGS. 5C & D are in patients with KS. A patient's genetic signature does not predict his/her ability to respond to kisspeptin. This is supported by Example 3 (See FIG. 5D), which showed that a patient who carried variants in two genes still responded. The present data supports a role of kisspeptin resistance as an over-arching pathomechanism for congenital hypogonadotropism despite a variety of underlying genetic diagnoses, which may be extended to other causes of reproductive endocrine dysfunction including hypothalamic amenorrhea, e.g., due to states of negative energy balance (e.g., undernourished, anorexic, or athletes), hyperprolactinemia, adult-onset hypogonadotropic hypogonadism, medication effects (e.g., from glucocorticoids, opioids) or attenuated forms of pathologic hypogonadotropic hypogonadism and/or those at risk for developing pathologic HH, e.g., subjects with idiopathic infertility, irregular menstrual cycles, or abnormal semen analysis. Sensitivity to kisspeptin increases with repetitive exposure; thus in some embodiments, the present methods can include repeated administration of kisspeptin to increase sensitivity to the point where endogenous kisspeptin secretion produces meaningful responses, to induce reversal of the kisspeptin resistance.

Kisspeptin and Kisspeptin Analogs

As used herein, Kisspeptin refers to a family of neuropeptides that result from the cleavage of a 145-amino-acid precursor peptide that is encoded by the KISS1 gene (Lee et al. 1996, Ohtaki et al. 2001). In humans, the active form of kisspeptin is thought to be a 54-amino-acid peptide (Ohtaki et al. 2001, Terao et al. 2004). The present methods can include administration of the full-length kisspeptin (NP_002247.3), the 54-amino-acid peptide (kisspeptin-54 (KP54)), or kisspeptin-10, a 10-amino-acid peptide comprising the sequence YNWNSFGLRF (SEQ ID NO:1). Analogs of Kiss-10 are known in the art and include those described in Curtis et al., Am J Physiol Endocrinol Metab. 2010 February; 298(2): E296-E303; Gutiérrez-Pascual et al., Mol Pharmacol. 2009 July; 76(1):58-67; Niida et al., Bioorg Med Chem Lett. 2006 Jan. 1; 16(1):134-7; Orsini et al., J Med Chem. 2007 Feb. 8; 50(3):462-71; Roseweir et al., J Neurosci. 2009 Mar. 25; 29(12):3920-9. In some embodiments, the analog is [dY]¹KP-10. Analogs can include those in which conservative substitutions are made to sequence disclosed herein, e.g., one, two or three amino acid substitutions, wherein the analog retains agonistic activities to KISS1R. In some embodiments, the analog is a peptide compound in which, inter alia, a peptide bond between a glycine residue and the adjacent residue, located in a region near the C-terminus of the compound, is replaced by a disubstituted 1,2,3-triazole ring, e.g., as described in WO2014118318A1; a peptide compound as described in US20200172575; Compound 1, described in US 20200046797 U.S. Pat. Nos. 7,960,348 and 8,404,643; C6, described in Parker et al., Theriogenology. 2019 May; 130:111-119; KISS1-305; TAK-448 (Ac-D-Tyr-D-Trp-Asn-Thr-Phe-azaGly-Leu-Arg(Me)-Trp-NH2 (SEQ ID NO:2); see Decourt et al., Sci Rep. 2016; 6: 26908; MacLean et al., J Clin Endocrinol Metab. 2014 August; 99(8):E1445-53; Skorupskaite et al., Hum Reprod Update. 2014 July; 20(4): 485-500) (e.g., TAK-448 acetate); or TAK-683 (N-Acetyl-YWNTFGL{Met-R}W-NH2 (SEQ ID NO:3); see Kanai et al., J Reprod Dev. 2017 June; 63(3): 305-310); and compounds described in US 20160074320; WO200285399; WO2004060264; WO2004101747; WO2004063221; EP1604682; WO2005117939; and EP1464652. See also Matsui and Asami, Neuroendocrinology 2014; 99:49-60. Pharmaceutically acceptable salts thereof, pharmaceutical compositions containing kisspeptin or a kisspeptin analog, can be used in the methods herein.

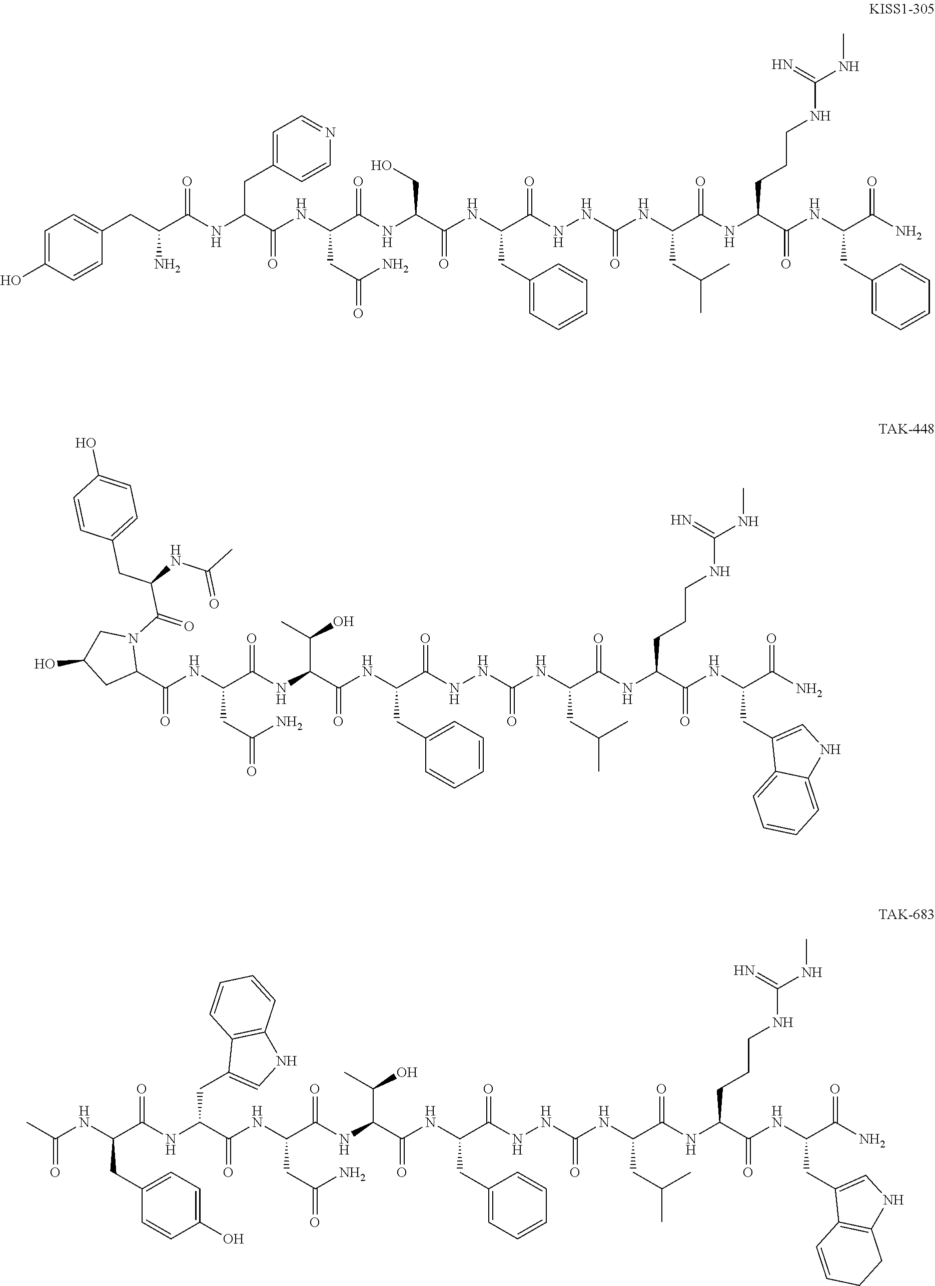

In some embodiments, where a peptide agonist is used, the peptide is modified.

Peptide analogs including reversed sequences can also be used, e.g., FRLGFSNWNY (SEQ ID NO:4).

Peptide analogs disclosed herein can include those modified according to methods known in the art for producing peptidomimetics. See, e.g., Qvit et al., Drug Discov Today. 2017 February; 22(2): 454-462; Farhadi and Hashemian, Drug Des Devel Ther. 2018; 12: 1239-1254; Avan et al., Chem. Soc. Rev., 2014, 43, 3575-3594; Pathak, et al., Indo American Journal of Pharmaceutical Research, 2015. 8; Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N terminus to the C terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetic include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as ∀-amino phosphonic acids and ∀-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. Exemplary retro-inverso peptidomimetics include FRLGFSNWNY, wherein the sequences include all D-amino acids.

The sequences can also be modified, e.g., by biotinylation or pegylation of the amino terminus and/or amidation of the carboxy terminus.

Opioid Antagonists

In some embodiments, the methods described herein include administration of an effective amount of an opioid antagonist, e.g., naloxone or naltrexone, or mixed agonists-antagonists, e.g., buprenorphine. Others can also be used, e.g., nalmefene. The opioid antagonist can be administered as an alternative to, or before, after, or concurrently with kisspeptin or a kisspeptin analog or other treatment as described herein. Thus provided herein are compositions comprising (i) kisspeptin or a kisspeptin analog and (ii) an opioid antagonist, e.g., naloxone or naltrexone, or a mixed agonist-antagonist, e.g., buprenorphine.

Conventional Treatments for Pathologic Hypogonadotropic Hypogonadotropism

In some embodiments, the methods include administration of gonadal steroid replacement therapy, including testosterone in males (e.g., testosterone esters (eg, enanthate, cypionate, undecanoate) and estrogen and/or progestin in females (e.g., conjugated estrogens (Premarin), ethinyl estradiol, or estradiol; and/or medroxyprogesterone, micronized progesterone); and/or hormonal contraceptives (e.g., ethinyl-estradiol/norethindrone). The methods can also include administration of gonadotropins, e.g., follicle stimulating hormone (FSH) or human chorionic gonadotropin (hCG). Optionally clomiphene and/or letrozole can be used to stimulate ovulation in a subset of patients.

### EXAMPLES

The invention is further described in the following examples, which do not limit the scope of the invention described in the claims.

### Example 1. Using Kisspeptin to Predict Pubertal Outcomes for Youth with Pubertal Delay

Materials and Methods

The following materials and methods were used in this Example.

Study Approval

The physiologic study on the kisspeptin-stimulation test was approved by the Massachusetts General Hospital (MGH)/Partners IRB and is registered on ClinicalTrials.gov (NCT01438034). Kisspeptin was used under IND 113,591, and GnRH was used under IND 93,353. Studies on the genetics of delayed puberty and IHH were approved by Boston Children's Hospital and MGH, respectively. All participants gave written informed assent, and at least one parent gave written informed consent. For the two participants who underwent retesting, the repeat testing was approved by the Partners IRB, and participants and their parents gave written informed assent and consent for the retesting.

Study Protocol

Details of the study protocol have been described previously (20). Briefly, inclusion criteria were age 12 years or older for girls and 13.5 years or older for boys, with delayed or stalled puberty defined based on breast development for girls and testicular volume for boys. Children with an identifiable cause of delayed puberty were excluded. Participants had two admissions to the MGH Translational and Clinical Research Center (TCRC) for blood sampling every 10 minutes to measure baseline LH secretion overnight, LH secretion in response to kisspeptin-10 0.313 mcg/kg (ΔLHₖᵢₛₛₚₑₚₜᵢₙ), and LH secretion in response to GnRH 75 ng/kg (ΔLH_{GnRH}). ΔLHₖᵢₛₛₚₑₚₜᵢₙ and ΔLH_{GnRH} were assessed both before and after pituitary “priming” with pulsatile subcutaneous GnRH 75 ng/kg every 2 hours for 6 days to ensure robust pituitary responsiveness to GnRH (8).

Participants subsequently returned every 6 months for follow-up visits to undergo physical examinations and measurement of FSH, LH, and either estradiol or testosterone to assess reproductive endocrine activity. Upon reaching the age of 18 years, participants underwent a final evaluation for physical and laboratory signs of reproductive endocrine activity. For participants receiving sex-steroid treatment, treatment was held prior to laboratory studies to ensure that sex-steroid measurements reflected endogenous production rather than exogenous administration (for 4 weeks for estradiol, for 6 weeks for injected testosterone, and for 2 weeks for transdermal testosterone).

Laboratory Assays

For the TCRC visits, LH, FSH, testosterone (T), and estradiol were measured by the MGH Clinical Laboratory Research Core, the Brigham and Women's Hospital Research Assay & Analysis Core, and Labcorp as previously described (20), e.g., using microparticle enzyme immunoassay using the automated Abbott AxSYM system (Abbott Laboratories, Chicago, Ill.) for serum LH and FSH, the DPC Coat-A-Count RIA kit (Diagnostics Products Corporation, Los Angeles, Calif.) for serum T concentrations (60). Precision of the LH assay was 4.3-6.4%. Inhibin B was measured in a single batch by immunoassay by the University of Virginia Ligand Assay Core, with an intraassay coefficient of variation of 2.5%. For follow-up visits, laboratory studies were performed by Labcorp and Quest Diagnostics. For participants outside the Boston area, follow-up data was obtained through their routine clinical care from their local endocrinologists and clinical laboratories.

Genetic Testing

Whole-exome sequencing was performed at the Broad Institute (Cambridge, Mass.). Exome-sequencing data was screened for variants in 30 genes associated with IHH/KS, delayed puberty, or both (listed in Table 1 (21)). Variants were classified according to criteria of the American College of Medical Genetics and Genomics (22).

Statistics

Fisher's exact test was used to assess the association between results of the kisspeptin-stimulation test and pubertal outcomes. A p-value <0.05 was considered significant. The Jeffreys interval was used to calculate confidence intervals for sensitivity and specificity.

TABLE 1
Genes associated with idiopathic hypogonadotropic hypogonadism,
Kallmann syndrome, and constitutional delay that were analyzed
in participants who underwent whole-exome sequencing

| Gene | Locus |
|---|---|
| ANOS1 (KAL1) | Xp22.32 |
| AXL | 19q13.2 |
| CHD7 | 8q12.2 |
| DMXL2 | 15q21.2 |
| DUSP6 | 12q21.33 |
| FEZF1 | 7q31.32 |
| FGF17 | 8p21.3 |
| FGF8 | 10q24.32 |
| FGFR1 | 8p12 |
| FLRT3 | 20p11 |
| GNRH1 | 8p21.2 |
| GNRHR | 4q21.2 |
| HS6ST1 | 2q21 |
| IGSF10 | 3q25.1 |
| IL17RD | 3p21.1 |
| KISS1 | 1q32 |
| KISS1R | 19p13.3 |
| NSMF (NELF) | 9q34.3 |
| PNPLA6 | 19p13.2 |
| POLR3A | 10q22.3 |
| POLR3B | 12q24 |
| PROK2 | 3p21.1 |
| PROKR2 | 20p12.3 |
| RNF216 | 7p22.1 |
| SEMA3A | 7q12.1 |
| SEMA3E | 7q21.11 |
| SOX10 | 22q13.1 |
| SPRY4 | 5q31.3 |
| TAC3 | 12q13.3 |
| TACR3 | 4q25 |
| WDR11 | 10q26 |

TABLE 2
Participant characteristics at screening

| | | | | | | Tanner | Right/Left | |
|---|---|---|---|---|---|---|---|---|
| | | Age | Height | Weight | BMI | Breast | Testicular | Sense of |
| ID | Sex | (y) | (cm) | (kg) | (kg/m²) | Stage | Volumes (mL) | Smell* |
| 1 | M | 17.0 | 163.8 | 66.7 | 24.9 | | 1/1 | self-reported |
| | | | | | | | | anosmia |
| 2 | M | 15.7 | 171.0 | 79.8 | 27.4 | | 3/3 | anosmia |
| 3 | F | 16.7 | 167.6 | 98.4 | 35.0 | I | | anosmia |
| 4 | M | 15.6 | 171.0 | 79.8 | 27.4 | | 2/1 | anosmia |
| A | M | 16.4 | 164.1 | 55.0 | 20.4 | | 2/ | anosmia |
| | | | | | | | impalpable | |
| 5 | F | 15.2 | 153.7 | 43.4 | 18.4 | I | | severe microsmia |
| 6 | F | 16.5 | 165.4 | 55.4 | 20.3 | I | | normosmia |
| 7 | M | 14.9 | 165.1 | 52.9 | 19.4 | | 1-2/1-2 | mild microsmia |
| 8 | M | 14.6 | 165.7 | 67.7 | 24.7 | | 2/2 | anosmia |
| 9 | M | 16.0 | 164.9 | 51.5 | 18.9 | | 6/6^{†} | mild microsmia |
| 10 | M | 14.9 | 149.9 | 49.5 | 22.1 | | 3/3 | normosmia |
| B | F | 15.0 | 154.5 | 64.7 | 27.1 | II^{†} | | ND |
| 11 | M | 15.1 | 167.0 | 53.2 | 19.1 | | 4/4^{†} | mild microsmia |
| 12 | M | 14.8 | 152.4 | 43.5 | 18.7 | | 1/1 | mild microsmia |
| 13 | F | 13.9 | 146.1 | 39.9 | 18.8 | I | | normosmia |
| 14 | M | 17.5 | 175.4 | 101.6 | 33.0 | | 8/8^{†} | severe microsmia |
| 15 | M | 14.2 | 153.0 | 38.9 | 16.6 | | 1/1 | normosmia |

| | Bone Age | | |
|---|---|---|---|
| | (Chronological Age | | Other |
| ID | When Assessed, y) | | Features |
| 1 | 14 | (16.5) | |
| 2 | 14 | (15.6) | |
| 3 | ND | | |
| 4 | 14 | (15.6) | synkinesia, unilateral renal |
| | | | agenesis |
| A | 14 | (16.4) | cryptorchidism |
| 5 | 13 | (15.0) | |
| 6 | 13 | (16.6) | |
| 7 | 13 | (14.9) | micropenis |
| 8 | 13 | (14.4) | micropenis, bilateral |
| | | | cryptorchidism |
| 9 | 13.5 | (15.3) | bilateral inguinal hernia |
| 10 | 13 | (13.8) | |
| B | 11 | (13.8) | |
| 11 | 12.5 | (14.5) | |
| 12 | 13 | (14.7) | history of traumatic birth, |
| | | | ventriculomegaly, seizures, |
| | | | bilateral cryptorchidism; |
| | | | unilateral renal cyst; |
| | | | leg-length discrepancy |
| 13 | 11 | (13.0) | |
| 14 | 14 | (14.7) | |
| 15 | 11 | (13.5) | |

TABLE 3
Follow-up findings

| | | Age | | | | Testi- |
|---|---|---|---|---|---|---|
| | | at Which | Ages of | Age at | | cular |
| | | Spontaneous | Sex- | Latest | | Volume |
| | | Pubertal | Steroid | Follow- | Tanner | (right/ |
| | | Progression | Treatment | Up | Breast | left, |
| ID | Sex | Noted (y) | (y) | (y) | Stage | mL) |
| 1 | M | — | 17.0- | 18.0 | | 2/ |
| | | | | | | impal- |
| | | | | | | pable |
| 2 | M | — | 15.3- | 18.6 | | ND |
| 3 | F | — | 16.9- | 18.0 | IV | |
| 4 | M | — | 14.7- | 18.1 | | 3/1 |
| A | M | — | 16.9- | 17.8 | | 3/ |
| | | | | | | impal- |
| | | | | | | pable |
| 5 | F | lost to F/U | | | | |
| 6 | F | — | 16.7- | 18.4 | II | |
| 7 | M | — | 15.9- | 18.3 | | 1/1 |
| 8 | M | — | 14.9- | 18.0 | | 3/3 |
| 9 | M | 16.6 | 15.9-16.3 | 18.9 | | 15/15 |
| 10 | M | 15.8 | none | 17.2 | | 8/8* |
| B | F | 16.3 | 15.4-15.7 | 16.3 | IV | |
| 11 | M | 16.0 | 14.5-15.4 | 18.0 | | 12/15 |
| 12 | M | 16.6 | 15.7-16.0 | 18.1 | | 12/12 |
| 13 | F | 14.6 | none | 18.1 | V | |
| 14 | M | 18.2 | 15.4-15.9, | 18.2 | | 12/12 |
| | | | 17.0-18.0 | | | |
| 15 | M | 17.5 | 14.4-17.0 | 18.0 | | 12-15/12 |

| | FSH | LH | Estradiol | Testosterone |
|---|---|---|---|---|
| ID | (mIU/mL) | (mIU/mL) | (pg/mL) | (ng/mL) |
| 1 | <0.2 | 0.1 | | 49 |
| 2 | <0.2 | 0.2 | | 9 |
| 3 | 0.2 | <0.2 | <5 | |
| 4 | 0.4 | 0.2 | | 22 |
| A | 0.2 | 0 | | 40 |
| 5 | | | | |
| 6 | 0.3 | 0.1 | <10 | |
| 7 | 0.8 | 0.4 | | 10 |
| 8 | 1.0 | 2.1 | | 50 |
| 9 | 2.5 | 2.9 | | 297 |
| 10 | 0.6 | 1.5 | | 186 |
| B | 2.9 | 1.3 | <20 | |
| 11 | 5.5 | 4.1 | | 422 |
| 12 | 14.4 | 5.7 | | 686 |
| 13 | 17.4 | 31.5 | 201 | |
| 14 | 4.0 | 2.9 | | 363 |
| 15 | 11.1 | 2.9 | | 398 |

### Results

Responses to Kisspeptin

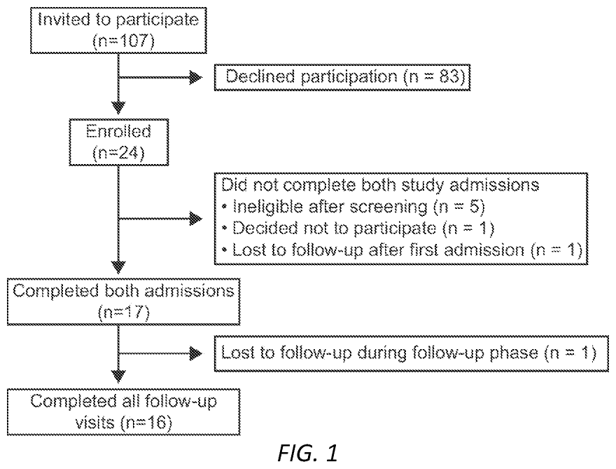

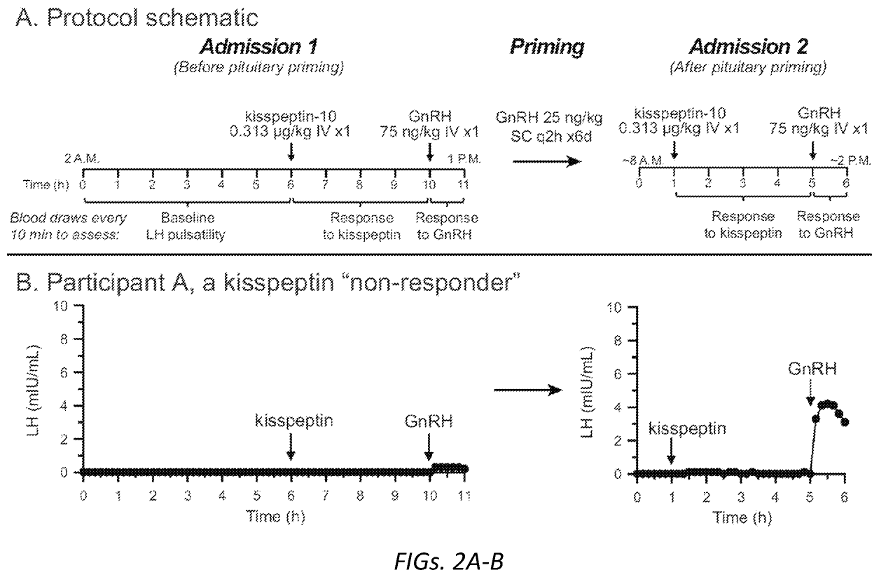

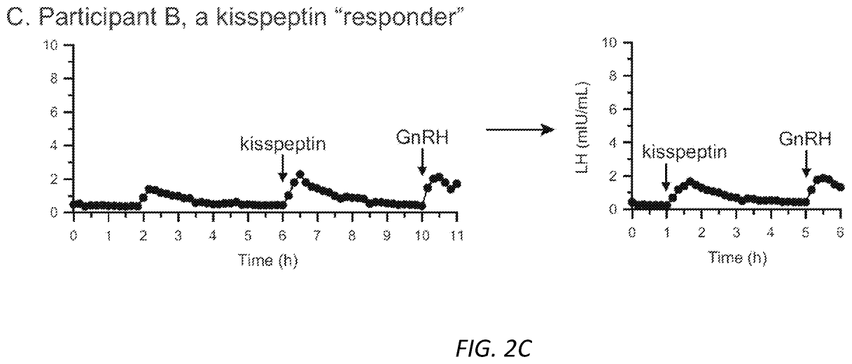

Seventeen participants with delayed or stalled puberty participated in this study (4 girls and 13 boys, FIG. 1). Results of kisspeptin- and GnRH-stimulation testing for 15 of these participants (Participants 1-15) were previously reported in ref. (20). Participant A, new to this study, demonstrated a “kisspeptin nonresponder” pattern, whereas Participant B, also new to this study, demonstrated a “kisspeptin responder” pattern (FIG. 2). Characteristics of the participants and their neuroendocrine phenotypes are provided in Table 4 and Table 2 (21).

Long-Term Follow-Up

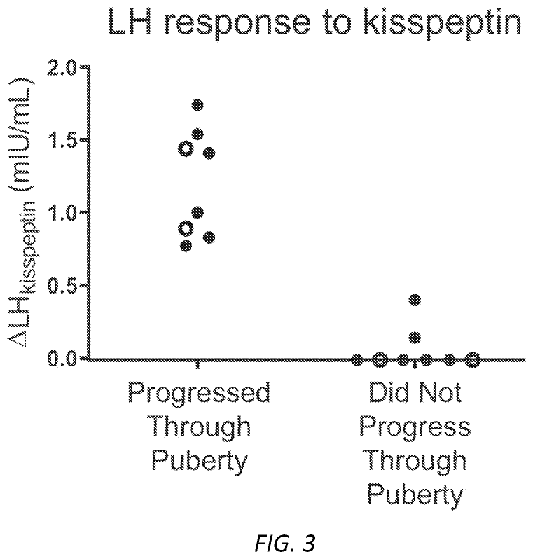

After this initial neuroendocrine characterization, participants returned for follow-up visits every 6 months. Eight participants (2 girls and 6 boys) progressed through puberty during the follow-up period (Table 4, Table 3 (21)). The boys demonstrated progressive increases in testicular volume, and the girls exhibited progressive breast development in the absence of exogenous treatment. All of these participants were “kisspeptin responders” who had responded to exogenous kisspeptin with a rise in LH of 0.8 mIU/mL or greater (FIG. 3, Table 4).

In contrast, 8 participants (1 girl and 7 boys) failed to enter puberty by age 18 years (Table 4, Table 3 (21)). These participants exhibited persistent sexual immaturity on physical examination; boys had testicular volumes <4 mL, and the girl did not have breast development until she started treatment with exogenous estradiol. On assessment of endogenous reproductive endocrine activity after discontinuation of sex-steroid treatment, all of these participants had serum concentrations of sex steroids (estradiol for girls, testosterone for boys) that were below the adult reference range and serum gonadotropin concentrations that were either low or inappropriately normal in the context of low sex steroids. These individuals who failed to enter puberty were “kisspeptin nonresponders” (1 girl and 6 boys) who had shown little to no response to kisspeptin (an eighth “nonresponder” was lost to follow-up) and 1 male “intermediate responder” who had shown an LH response to kisspeptin of 0.4 mIU/mL (FIG. 3, Table 4).

Thus, the participants' responses to kisspeptin cleanly distinguished those who later progressed through puberty from those who did not (p=0.0002). Sensitivity and specificity for the kisspeptin-stimulation test were both 100% (95% CI 74-100%).

LH Measurements Under Three Conditions: Daytime, Overnight, and after GnRH Stimulation

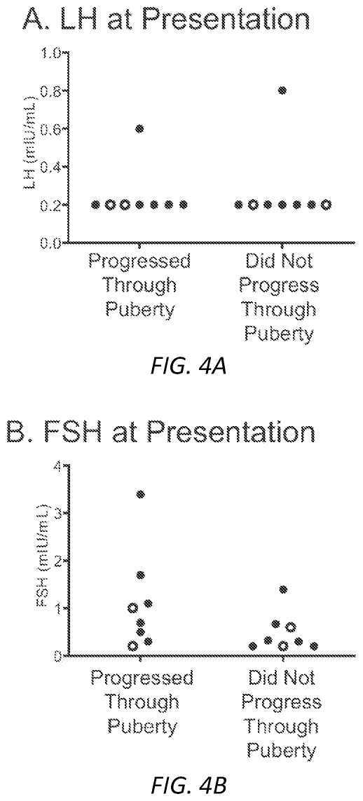

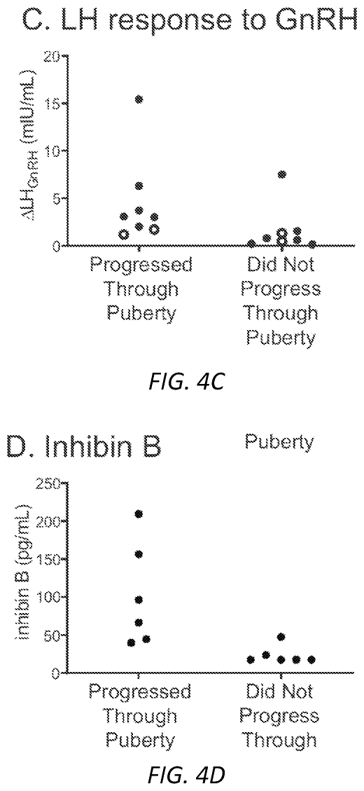

Unstimulated LH concentrations (measured during the day or overnight) and LH measured after stimulation by exogenous GnRH have been studied as methods to predict whether a child will eventually enter puberty (8). In this study, 2 boys had unstimulated serum LH concentrations in the pubertal range at the time of enrollment, and 1 was later found to progress through puberty. Of the remaining 14 participants with LH in the prepubertal range at enrollment, 7 later progressed through puberty and 7 did not (FIG. 4, Table 4). Thus, unstimulated LH did not accurately predict pubertal outcomes.

In early puberty, daytime gonadotropin measurements may not accurately reflect activity of the reproductive endocrine axis because pulses of GnRH and LH secretion occur only at night during the deep stages of sleep (23). We had therefore conducted overnight blood sampling to detect sleep-associated LH secretion. All 6 children who had at least one pulse of LH secretion overnight were later found to progress through puberty (Table 4). In contrast, variable outcomes were observed for the 10 children who had no LH pulses overnight. While 8 failed to progress through puberty, 2 exhibited pubertal progression: one girl and one boy (Participants B and 11) who at the time of neuroendocrine evaluation were being treated with exogenous sex steroids that may have suppressed endogenous LH secretion.

GnRH-stimulated LH secretion has also been studied as a test to predict pubertal outcomes (8). In our cohort, LH responses to GnRH were overlapping, ranging from 1.2 to 15.4 mIU/mL in those who later progressed through puberty and from 0.2 to 7.5 mIU/mL in those who did not (FIG. 4, Table 4).

Thus, none of these tests—unstimulated LH, whether measured during the day or at night, or GnRH-stimulated LH—was as accurate as the kisspeptin-stimulation test for predicting pubertal outcomes in this study cohort.

Inhibin B

Measurement of serum inhibin B has been proposed as a method to predict pubertal outcomes for children with delayed puberty (8-11). Serum inhibin B ranged from 39 to 209 pg/mL in boys who later progressed through puberty and from <17 pg/mL to 48 pg/mL in boys who did not (FIG. 4, Table 4). Thus, inhibin B did not accurately distinguish those who would later progress through puberty from those who would not.

Genetic Testing

A subset of participants consented to exome sequencing. Pathogenic and likely pathogenic variants in 30 DM genes (Table 1; (21)) were identified in 2 of 5 participants who later progressed through puberty and 2 of 6 participants who did not enter puberty (Table 5). No pathogenic or likely pathogenic variants were identified in IGSF10, a gene potentially associated with constitutional delay. Thus, genetic testing could not predict pubertal outcomes for children with pubertal delay.

TABLE 4
Participant characteristics and reproductive endocrine evaluation

| | | | | | | Before pituitary priming | | | After | |
|---|---|---|---|---|---|---|---|---|---|---|
| | | At enrollment | | | | Over- | | | pituitary | Follow-up |
| | | | Pubertal | | | night | Inhibin | | priming | Pubertal |
| ID | | Age | develop- | LH | FSH | LH | B | ΔLH_{GnRH} | ΔLHₖᵢₛₛₚₑₚₜᵢₙ | Progres- |
| * | Sex | (y) | ment | (mIU/mL) | (mIU/mL) | pulses | (pg/mL) | (mIU/mL) | (mIU/mL) | sion? |
| 1 | M | 17.0 | Absent | 0.1 | 0.2 | Absent | <17 | 0.2 | 0 | N |
| 2 | M | 15.7 | Absent | 0.2 | 0.2 | Absent | <17 | 0.2 | 0 | N |
| 3 | F | 16.7 | Absent | <0.2 | 0.2 | Absent | ND | 0.5 | 0 | N |
| 4 | M | 15.6 | Absent | 0.1 | 0.7 | Absent | <17 | 0.6 | 0 | N |
| A | M | 16.4 | Absent | <0.1 | 0.3 | Absent | 23 | 0.7 | 0 | N |
| 5 | F | 15.2 | Absent | 0.1 | 0.5 | Absent | ND | 0.8 | 0 | Lost to |
| | | | | | | | | | | follow-up |
| 6 | F | 16.5 | Absent | <0.2 | 0.6 | Absent | ND | 1.3 | 0 | N |
| 7 | M | 14.9 | Absent | 0.1 | 0.3 | Absent | <17 | 1.6 | 0.1 | N |
| 8 | M | 14.6 | Absent | 0.8 | 1.4 | Absent | 47 | 7.5 | 0.4 | N |
| 9 | M | 16.0 | Stalled | 0.1 | 0.5 | Present | 209 | 3.7 | 0.8 | Y |
| 10 | M | 14.9 | Absent | <0.3 | 1.1 | Present | 96 | 3.0 | 0.8 | Y |
| B | F | 15.0 | Absent | 0.1 | 0.2 | Absent^{†} | ND | 1.2 | 0.9 | Y |
| 11 | M | 15.1 | Stalled | 0.1 | 0.3 | Absent^{†} | 156 | 6.3 | 1.0 | Y |
| 12 | M | 14.8 | Absent | 0.1 | 1.7 | Present | 44 | 3.1 | 1.4 | Y |
| 13 | F | 13.9 | Absent | 0.1 | 1.0 | Present | ND | 1.7 | 1.4 | Y |
| 14 | M | 17.5 | Stalled | 0.2 | 0.7 | Present | 66 | 15.4 | 1.5 | Y |
| 15 | M | 14.2 | Absent | 0.6 | 3.4 | Present | 39 | 2.0 | 1.7 | Y |

*Participants with numerical ID’s are numbered as in ref. (20); participants with alphabetical ID’s enrolled in the study after that report. ^{†}Participants B and 11 were receiving exogenous sex steroids at the time of their pre-priming visits. ΔLHₖᵢₛₛₚₑₚₜᵢₙ, increase in luteinizing hormone (LH) after kisspeptin; ΔLH_{GnRH}, increase in LH after gonadotropin-releasing hormone (GnRH). ND, not determined. Some data in this table were previously reported in ref. (20).

TABLE 5
Pathogenic and likely pathogenic variants in genes associated with
idiopathic hypogonadotropic hypogonadism

| | | Pubertal | |
|---|---|---|---|
| ID | Sex | Progression? | Variant Identified |
| 1 | M | N | ND |
| 2 | M | N | No qualifying variants found |
| 3 | F | N | FGFR1 p.R609X |
| 4 | M | N | ANOS1* p.T193Kfs*24 |
| A | M | N | No qualifying variants found |
| 6 | F | N | No qualifying variants found |
| 7 | M | N | ND |
| 8 | M | N | No qualifying variants found |
| 9 | M | Y | ND |
| 10 | M | Y | No qualifying variants found |
| B | F | Y | ND |
| 11 | M | Y | No qualifying variants found |
| 12 | M | Y | FGFR1 p.I676Dfs*7 |
| 13 | F | Y | No qualifying variants found |
| 14 | M | Y | ND |
| 15 | M | Y | TACR3 p.W208X |

*Formerly named KAL1. ND, not done.

### Example 2. Supraphysiologic Kisspeptin to Improve Pubertal Outcomes for Youth with Pubertal Delay

One subject with pubertal delay in the study described in Example 1 received kisspeptin-10 0.313 μg/kg WB (physiologic dose) at age 15.3 y and failed to mount a LH response. During a separate admission shortly thereafter, he received a supraphysiologic dose of kisspeptin (18 μg/kg), and surprisingly, did respond. The subject's final diagnosis was IHH as evidenced by failure to show physical signs of puberty by 18 y. This demonstrates that 1) GnRH deficiency can be attributed to kisspeptin resistance and 2) this resistance can be overcome with high-dose kisspeptin.

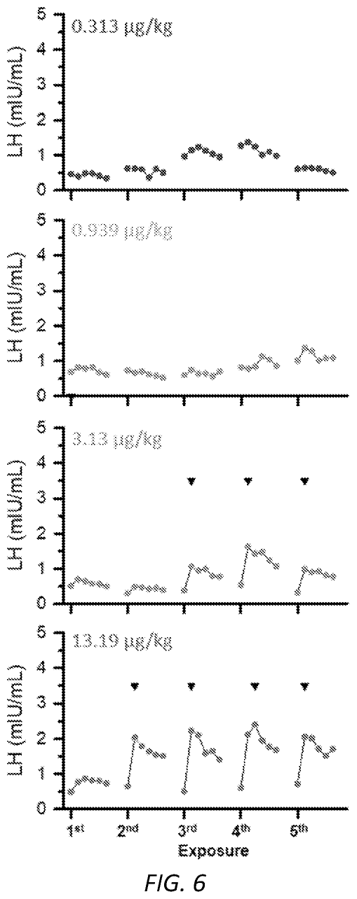

Based on this observation, it was hypothesized that high-dose and/or repetitive administration of kisspeptin could stimulate GnRH-induced LH secretion in patients with IHH/KS. To address this hypothesis, the following protocol was initiated: 1) q10 min blood sampling×6 h, 2) kisspeptin administration q2 h×40 h (5 sets of 4 doses each: 0.313, 0.939, 3.13, and 13.19 μg/kg). For comparison, FIG. 5A shows a healthy male. He has normal LH pulses and a physiologic dose of kisspeptin clearly resulted in a GnRH-induced LH pulse. FIG. 5B shows a KS male who has no endogenous LH pulses and no LH response to kisspeptin (i.e., kisspeptin resistance). He carries a rare variant in the gene encoding the prokineticin receptor, PROKR2, and DMXL2. FIG. 5C shows a KS female who did not respond to the physiologic dose of kisspeptin, but did respond to supraphysiologic doses with clear kisspeptin-induced GnRH-induced LH pulses. Moreover, the amplitude of the pulses increased over time, suggesting a “priming” effect of repetitive exposure. Her genetic signature is not yet known. FIG. 5D shows a KS male also with genetic variants, who also responded to supraphysiologic kisspeptin, although his response is not as pronounced. FIG. 6 shows the data from FIG. 5C, re-graphed to clearly show the responses to each dose over time. Thus, although patients with abiding hypogonadotropism were thought to be kisspeptin resistant, at a single IV bolus dose, these patients demonstrated a very different response when kisspeptin was administered repetitively, particularly at higher doses. Their data demonstrates that GnRH neurons in patients with IHH/KS could be awakened from their state of quiescence by repetitive kisspeptin stimulation. Moreover, this repetitive kisspeptin stimulation resulted in GnRH-induced LH pulsations.

### Example 3. Hypothalamic Reproductive Endocrine Pulse Generator Activity Independent of Neurokinin B and Dynorphin Signaling

Identification of the afferent pathways through which endogenous factors (e.g. gonadal steroids, stress hormones, and nutrient signals) and external cues (e.g. social cues and day length) regulate GnRH release have recently focused on the kisspeptin/neurokinin B/dynorphin system (34). Inactivating mutations in kisspeptin, neurokinin B (NKB), and their respective receptors cause DM in humans and mice, implicating these neuropeptides in the generation of GnRH pulses (35-42). Dynorphin is thought to oppose this stimulatory activity by providing critical slowing of GnRH pulse generator activity in response to progesterone during the luteal phase of the menstrual cycle (43-45). These three neuropeptides coalesce in a population of neurons in the arcuate nucleus, KNDy (Kisspeptin-Neurokinin B-Dynorphin) neurons, and are postulated to work in a coordinated fashion to synchronize the secretory activity of GnRH neurons to generate the pulses of GnRH secretion that are necessary to drive reproductive endocrine function (46-48).

Because biallelic loss-of-function mutations disrupt both copies of a gene, patients carrying such mutations (i.e. “human knockouts”) provide novel insights into the phenotypic consequences of gene disruption or loss. In this study, four sisters carrying biallelic, complete loss-of-function mutations in the gene encoding NKB (one of the key neuropeptides in KNDy neurons) underwent genotype-driven phenotyping. Despite an initial diagnosis of IHH, several sisters spontaneously recovered reproductive endocrine function in adult life. Studies were performed in both normal and neurokinin B-deficient family members as well as normal and neurokinin B-deficient mice to investigate the role of NKB in GnRH pulse generation and to dissect the interactions between NKB, kisspeptin, and dynorphin. Use of a combination of specific neuroendocrine probes revealed that the hypothalamus is capable of generating GnRH-induced LH pulses despite genetic and pharmacologic antagonism of two of the three KNDy constituents, NKB and dynorphin.

The results shows an increase in LH levels during the NLX infusion in subjects with IHH. To date, the ability to stimulate endogenous GnRH-induced LH pulsations that mimic normal physiology in patients with IHH has been non-existent.

Considerations regarding LH pulses include the observation that Subject 4 appeared to have a more pronounced response to NLX than Subject 5. Subject 4 underwent pituitary priming with exogenous GnRH and Subject 5 did not, which may have amplified any effect of NLX on the LH response in Subject 4. Subject 4 had also been receiving intermittent hormone replacement therapy which may have enhanced endogenous kisspeptin action on GnRH release.

In prior studies, the inability of the same dose of kp-10, which effects a robust GnRH-induced LH response in healthy men and luteal-phase women, to bring about any effect in IHH patients across a range of genotypes suggested that the functional capacity of the GnRH neuronal network is fundamentally impaired in patients with IHH (58). In contrast to these previous observations in IHH patients with genotypes other than TAC3 or TACR3, Subjects 3, 4, and 5 responded to kp-10 IVB (58). Here, the low frequency pulses and the ability to respond to exogenous kp-10 administration suggest that the GnRH neuronal circuitry necessary for pulse generation remains intact in patients lacking NKB. However, the ability to respond to kp-10 with LH pulses was observed only in the setting of IVB administration, and not a continuous infusion, as has been reported by others (59). Differing doses of kp-10, LH assays and LH pulse algorithms may account for this discordance.

Methods

The following Materials and Methods were used in Example 3.

Subjects and Eligibility Criteria

Five women from a single consanguineous family were recruited on the basis of their genotype (Table 6). Subjects were either reproductively normal (Subject 1; genotype TAC3 c.61_61delG p.A21LfsX44 heterozygote) or carried a diagnosis of hypogonadotropic hypogonadism (Subjects 2-5; genotype TAC3 c.61_61delG p.A21LfsX44 homozygote). The brothers and parents were not available for study participation. IHH was defined as hypogonadal sex steroid levels (estradiol <20 pg/mL in women) in the setting of low or normal gonadotropin levels at age ≥18 years and the absence of any identifiable medical condition that could cause hypogonadotropic hypogonadism. As in our previous report (19), reversal of IHH in women was defined as: 1) fertility without use of exogenous GnRH or gonadotropin therapy; 2) spontaneous menstrual cycling for at least 3 months in the absence of treatment; and/or 3) LH pulse frequency and amplitude within the normal range for women. Relapse after reversal was defined as again having hypogonadal sex-steroid levels (serum estradiol <20 pg/mL in women) and/or amenorrhea.

Subjects also participated in a genetics study. Patient DNA was screened for rare sequence variants (RSVs), defined as having a minor allele frequency of less than 1% in The Genome Aggregation Database (gnomAD), in genes known to cause IHH, as described previously (20, 21). Genes screened were CHD7 (MIM 608892), FGF8 (MIM 600483), FGFR1 (MIM 136350), GNRH1 (MIM 152760), GNRHR (MIM 138850), HS6ST1 (MIM 604846), ANOS1 (previously called KAL1, MIM 300836), KISS1 (MIM 603286), KISS1R (MIM 604161), NSMF (previously called NELF, MIM 60813), PROK2 (MIM 607002), PROKR2 (MIM 607123), TAC3 (MIM 162330), and TACR3 (MIM 162332) by PCR amplification of exons followed by Sanger sequencing. RSVs were reported if they were predicted to be damaging by at least 2 out of 4 in silico prediction programs: PolyPhen-2 (22), SIFT (23), Mutation Taster (24), or Panther (25). The University of Pennsylvania Smell Identification Test (UPSIT) scores, from a 12-item smell test, were used to classify olfactory capabilities (26, 27).

TABLE 6
Study Subject Characteristics.

| | | |
|---|---|---|
| ID | Presentation | Initial Treatment and Subsequent Course |

TAC3 c.61_61delG p.A21LfsX44 heterozygote

| | | |
|---|---|---|
| 1 | 12.6 y, | 12.6 y-35 y, regular monthly menses |
| | menarche | 35 y, pregnant |

TAC3 c.61_61delG p.A21LfsX44 homozygote

| 2 | 15 y, 10° | | 15-20 y, HRT with breast development, growth spurt | | | |
|---|---|---|---|---|---|---|
| | amenorrhea, | | 20 y, MPA X 10 d + withdrawal bleed | | | |
| | minimal | | mid-20s, HRT × 6 mo | | | |
| | thelarche | | mid-20s, herbal medication | | | |
| | | | 31 y-present, amenorrheic | | | |
| 3 | 14 y, 1° | | 16 y 8 mo, FSH 2.1 IU/L (0.6-11), LH 1.1 IU/L (1-11), E₂ <40 pmol/L | | | |
| | amenorrhea, | | 16-20 y, HRT with breast development, growth spurt HRT | | | |
| | no thelache | | 22 y, FSH 7.7 IU/L (0.6-11), LH 11.9 IU/L (1-11) | | | |
| | | | 22 y, MPA x1 + withdrawal bleed | | | |
| | | | 22 y, spontaneous conception of healthy son, 1 most MPA | | | |
| | | | 24 y, superovulation ×2 (MPA followed by CC), no pregnancies | | | |
| | | | 24-37 y, ~ q3 mo MPA, + intermittent withdrawal bleeds | | | |
| | | | 37-40 y, amenorrheic | | | |
| | | | 40 y-present, restarted on ~ q3 mo MPA | | | |
| 4 | 14 y, 1° | | 16 y 4 mo, FSH 2.4 IU/L (0.6-11), LH <0.5 IU/L (1-11), E₂ 49 pmol/L | | | |
| | amenorrhea, | | 16-22 y, HRT with breast development | | | |
| | no thelache | | 22-24 y, amenorrheic | | | |
| | | | 24 y, + home pregnancy test followed by SAB | | | |
| | | | 24 y, FSH 5.5 IU/L (0.6-11), LH 5.4 IU/L (1-11) | | | |
| | | | 25-27 y, HRT | | | |
| | | | 28 y 5 mo, herbal medication, 2 spontaneous cycles 6 mo apart | | | |
| | | | 28 y, FSH, LH “normal range”, E₂ “low” at 52 pmol/L | | | |
| | | | 29-30 y, HRT | | | |
| | | | 30-31 y, amenorrheic | | | |
| | | | 31-present, intermittent HRT use | | | |
| 5 | 13 y, 1° | | 17-18 y, HRT | | | |
| | amenorrhea, | | 21 y, OCPs for 6 mo | | | |
| | no thelarche | | 25 y, herbal medication + withdrawal bleed, repeated without effect | | | |
| | | | 26-28 y, amenorrheic | | | |
| | | | 28-29 y, regular monthly cycling (1.3 y) | | | |
| | | | 29-31 y, q2.5 mo cycles (2.5 y) | | | |
| | | | 31 y-present, yearly spontaneous spotting | | | |
| | | Research Study 2016 | | | | |
| | | | FSH | LH | E₂ | |
| ID | Presentation | Protocol | (IU/L) | (IU/L) | (pg/mL) | Imaging |

TAC3 c.61_61delG p.A21LfsX44 heterozygote

| | | | | | | |
|---|---|---|---|---|---|---|
| 1 | 12.6 y, | 1) Baseline | 4.23 | 2.18 | 20.4 | US: endometrium 6 mm, multiple |
| | menarche | 2) IVB Kiss, GnRH | | | | small follicles |

TAC3 c.61_61delG p.A21LfsX44 homozygote

| 2 | 15 y, 1° | Not applicable | | | | |
|---|---|---|---|---|---|---|
| | amenorrhea, | | | | | |
| | minimal | | | | | |
| | thelarche | | | | | |
| 3 | 14 y, 1° | 1) Baseline & IVB Kiss, | 2.12 | 0.49 | 20.3 | normal MRI |
| | amenorrhea, | GnRH | | | | US-endometrium 4 mm, all |
| | no thelache | 2) Kisspeptin Infusion & | | | | follicles <2 mm, uterus small |
| | | IVB GnRH | | | | adult size |
| 4 | 14 y, 1° | 1) Baseline & IVB Kiss, | 3.97 | 0.94 | 11.2 | US-endometrium 5 mm, one |
| | amenorrhea, | GnRH | | | | follicle 10 mm, uterus small adult |
| | no thelache | 2) Naloxone Infusion & | | | | size |
| | | IVB Kiss, GnRH | | | | |
| 5 | 13 y, 1° | 1) Baseline & IVB Kiss, | 3.42 | 0.86 | 34 | normal MRI |
| | amenorrhea, | GnRH | | | | US-endometrium 9 mm, cyst |
| | no thelarche | 2) Naloxone Infusion & | | | | 3 cm |
| | | IVB Kiss, GnRH | | | | |

FSH = follicle stimulating hormone, LH = luteinizing hormone, E2 = estradiol, IVB = intravenous bolus, kiss = kisspeptin, GnRH = gonadotropin stimulating hormone, US = transvaginal ultrasound, HRT = hormone replacement therapy, MPA = medroxyprogesterone acetate, CC = clomiphene citrate, SAB = spontaneous abortion, OCPs = oral contraceptive pills

Study Design

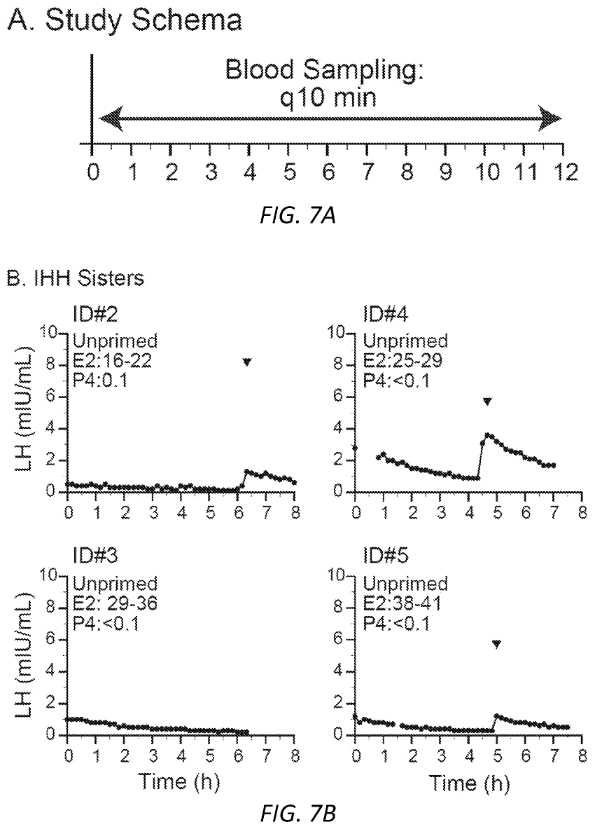

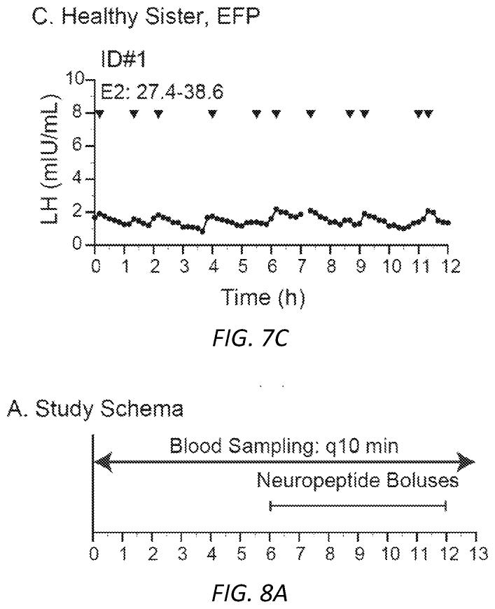

In 2010, the subjects with hypogonadotropic hypogonadism (Subjects 2, 3, 4, 5) underwent detailed neuroendocrine phenotyping in which blood sampling was performed every 10 minutes (q10 min) for 6-8 hours to map endogenous LH pulsations at the Wellcome Trust Clinical Research Facility, Cambridge, UK under the direction of Professor I. Sadaf Farooqi (FIG. 7A).

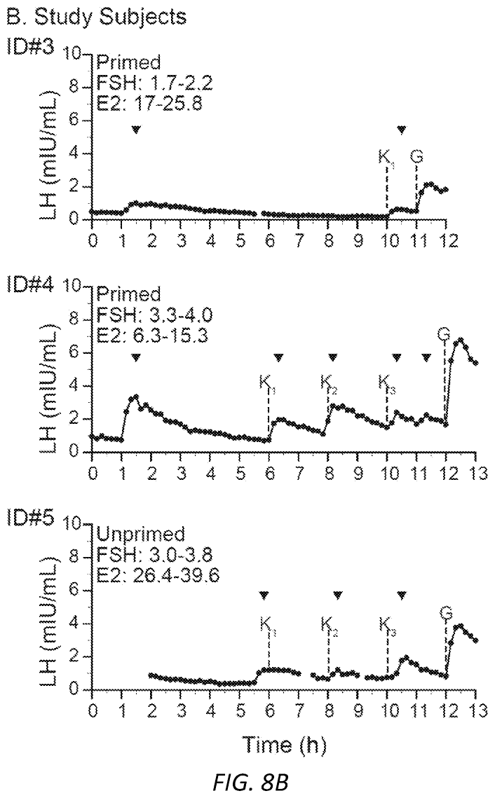

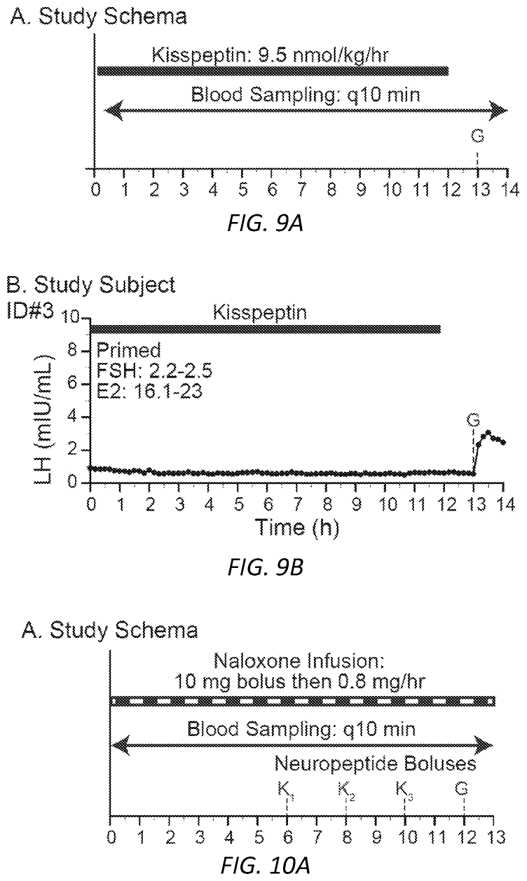

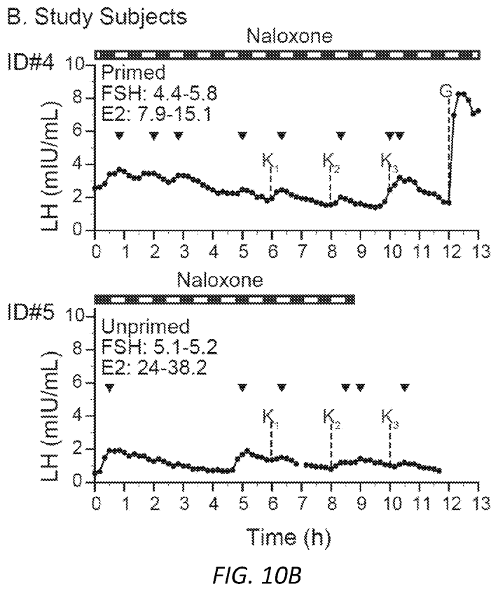

In 2016, Subjects 1, 3, 4, and 5 were invited to participate in a second series of daytime studies at Massachusetts General Hospital (MGH) Clinical Research Center (CRC) to determine whether their endogenous LH pulse patterns could be modified by administration of GnRH, kisspeptin 112-121 (kp-10) and the non-specific opioid antagonist which blocks dynorphin, naloxone (NLX) (FIG. 8A, FIG. 9A, FIG. 10A). To ensure that the pituitary gonadotropes would be in a state of readiness, Subjects 3 and 4 received exogenous pulsatile GnRH 25 ng/kg every 2 hours (q2 h) by a Crono F portable infusion pump (Canè S.p.A, Turin, Italy) for 3 days prior to admission to the MGH CRC (28). Subject 5 had recent evidence of some neuroendocrine activity (yearly spontaneous bleeding) so she was not primed with pulsatile GnRH (Table 6).

Baseline studies: All subjects underwent q10 min blood sampling for at least 6 hours to evaluate endogenous GnRH-induced LH secretion during one of their visit days to the MGH CRC (FIG. 7A, FIG. 8A).

Kisspeptin boluses: After assessment of endogenous GnRH-induced LH secretion, subjects 3, 4, and 5, received the administration of kp-10 0.24 nmol/kg intravenous bolus (IVB) as prior work by our group demonstrated that this dose consistently elicits GnRH-induced LH pulses of physiologic amplitude in healthy men and healthy luteal-phase women (29, 30) (FIG. 8A). Subjects 4, 5 received subsequent kp-10 IVBs of 0.72 and 2.4 nmol/kg. Subjects 3, 4, and 5 then received 75 ng/kg IVB of GnRH at the conclusion of these studies, as our group has previously shown that this dose results in robust GnRH-induced LH responses in individuals with intact gonadotrope function (31).

Kisspeptin Infusion: In contrast to the WB studies, Subject 3 returned to the CRC to participate in a second admission in which kp-10 was administered as a continuous infusion (9.5 nmol/kg/hr) for 12 hours to determine its effect on endogenous GnRH-induced LH pulsations. Similar to the WB studies, blood samples were drawn q10 min and GnRH 75 ng/kg IVB was administered at study conclusion (FIG. 9A).

Naloxone Infusion, Blocking Dynorphin: Subjects 4 and 5 returned to the CRC and received an NLX infusion (NLX 10 mg WB, followed by infusion at 0.8 mg/hr) for 13 hours to determine the effect of blocking dynorphin signaling with opioid antagonism on endogenous LH pulses in the absence of NKB signaling. Midway through the infusion, kp-10 and GnRH boluses (kp-10 dose range: 0.24 to 2.4 nmol/kg, GnRH: 75 ng/kg) were administered to determine whether NLX administration might enhance the response to these peptides (FIG. 10A). Again, blood samples were drawn q10 min for hormone measurements. Due to nursing error, subject 5 had the NLX infusion terminated early at hour 9.

Source of Peptides

Kisspeptin 112-121, the 10-amino-acid isoform of kisspeptin (corresponding to amino acids 112-121 of the pre-prohormone), and GnRH were synthesized using good manufacturing practices by NeoMPS (PolyPeptide Laboratories, San Diego, Calif.). NeoMPS provided kisspeptin 112-121 under contract to the Eunice Kennedy Shriver National Institute of Child Health and Human Development. Naloxone was ordered from Hospira (Lake Forest, Ill.).

Human Laboratory Assays

LH for each sample and estradiol on 2-hour pools were measured by direct immunoassay using the automated Abbott ARCHITECT system (Abbott Laboratories, Inc., Abbott Park, Ill.) as previously described (28). Estradiol was measured by a 2nd generation immunoassay traceable to mass spectrometry-based assays for the 2010-2011 studies and by Elecsys (Roche Diagnostics, Indianapolis, Ind.) for 2016 studies (32, 33).

Assessment of Pulsatile LH Release in Peripubertal and Adult Tac2 Knockout Mice

Tac2^{+/-} breeding pairs were generated by the Texas A&M Institute for Genomic Medicine (College Station, Tex.) and genotyped (34). All mice were generated and maintained on a Sv129/C57BL/6 hybrid background and group housed (three to five per cage) at the Brigham and Women's Hospital in a temperature- and light-controlled environment with lights on from 0600-1800 h and food and water provided ad libitum. Mice were handled daily for two to six weeks prior to the experiment to allow acclimation to sampling conditions.

Changes in LH secretion was assessed in sexually maturing (6-week-old) and adult (16-week-old) intact and ovariectomized (OVX) Tac2 knockout (KO) female mice and control (wild-type; WT) littermates (n=4-5 per group). Since Tac2 in mice encodes for NKB in humans, these mice are lacking NKB. Pulsatile measurements of LH secretion were assessed by repeated blood collection through a single incision at the tip of the tail. The tail was cleaned with saline then four ul blood was taken at each time point from the cut tail with a pipette. Whole blood was immediately diluted in 116 ul of 0.05% PBST, vortexed, and frozen on dry ice. Samples were stored at −80° C. for a subsequent LH ELISA. For kp-10 administration studies, thirty-six sequential blood samples were collected over a 6-hour sampling period. At 170 min of sampling (or 180 min of sampling for peripubertal Tac2 knockout mice), mice were injected with mouse kp-10 intraperitoneally (7.5 nmol/100 ul saline; Phoenix Pharmaceuticals). For NLX administration studies, thirty sequential blood samples were collected over a 5-hour sampling period from WT and Tac2 KO mice. WT and Tac2 KO mice were OVX'd to increase the frequency and amplitude of LH pulses to better determine the action of dynorphin removal in the generation of LH pulses. At 120 min of sampling, mice were injected with NLX intraperitoneally (5 mg/kg/100 ul saline; Sigma Aldrich).

Data Analysis

Human Pulse Analysis: LH pulses were identified using a validated modification of the Santen and Bardin method (35, 36) augmented by a deconvolution algorithm (29). Pulse amplitude of kp-10-induced or GnRH-induced LH pulses was calculated as the difference between time 0 of kp-10 or GnRH administration and the peak of the pulse.

Mouse Pulse Analysis: LH pulses were identified using a custom-made MATLAB code that reads the LH pulse data gathered by LH sandwich ELISA. The code includes a loop that determines a pulse based on if: a) the height of an LH value is 20% greater than the heights of either of the 2 previous values as well as 10% greater than the height of the following value; b) the peak at the second-time interval (i=2) is >20% greater than the single value that comes before it to be considered a pulse.

Statistics: Paired two-way t-tests were used to assess changes in mean LH, LH amplitude (nadir to peak of an LH pulse) and FSH at baseline, as defined in methods above, as compared to responses to neuropeptide interventions. All values are reports as mean±standard deviation, unless otherwise noted.

Study Approval

All human studies were approved by the Institutional Review Board of MGH/Partners Healthcare, or by the Local Regional Ethics Committee of Cambridge, United Kingdom. All subjects gave written informed consent prior to inclusion in the studies. For the mouse studies, the Brigham and Women's Hospital Institutional Animal Care and Use Committee approved all procedures.

Results

Study Subjects Initial Clinical Presentation and Subsequent Course

Subject 1 had a normal timing of menarche, normal menstrual cycles, and spontaneous pregnancy (Table 6). Her sisters, Subjects 2, 3, 4, and 5, presented at 13-15 y with primary amenorrhea and received estrogen therapy to induce secondary sexual characteristics. Because of the lack of spontaneous sexual maturation by age 18, normal MRI, and low gonadotropins, Subjects 2, 3, 4, and 5 all received a diagnosis of IHH (Table 6). None of the sisters are anosmic. Three of the four IHH sisters demonstrated reversal of their hypogonadotropism between 22-28 y as evidenced by pregnancy without fertility medications (Subjects 3 and 4) and regular spontaneous menstrual cycles (Subject 5). However, reversal was not permanent and at the time of the physiologic studies, subjects 3, 4, and 5 had reverted to a state of hypogonadotropic hypogonadism (Table 6).

Genetics

Sequencing of candidate genes revealed that Subject 1 (normal timing of puberty and normal menstrual cycles) is heterozygous for a deletion of a single nucleotide in the gene encoding NKB (TAC3) (c.61_61delG p.A21LfsX44). This base pair deletion leads to a frameshift mutation and a premature stop codon in the pre-prohormone prior to the NKB sequence that would be predicted to result in nonsense-mediated decay. Even if the transcript were to escape nonsense-mediated decay, the frameshift mutation would disrupt the portion of the pre-prohormone that is processed to produce the decapeptide known as NKB. Subjects 2, 3, 4, and 5, all with hypogonadotropic hypogonadism, are homozygous for this frameshift mutation. This mutation is novel and not found in gnomAD, a normative database containing 123,136 exomes and 15,496 genomes (21). Notably, there are no individuals homozygous for any protein-truncating mutations in TAC3 in gnomAD. This family harbors no other mutations in genes known to cause IHH.

Baseline Studies: Slow LH Pulse Frequency Characterizes IHH Individuals without Neurokinin B

At the time of these baseline studies, the IHH sisters (Subjects 2, 3, 4 and 5) were amenorrheic with low but detectable serum estradiol levels and low progesterone levels off hormonal medications (Table 6, FIG. 7B). All subjects with IHH had evidence of an enfeebled but organized GnRH pulse generator, as evidenced by low-frequency LH secretory events (for comparison in the physiologic early follicular phase which is characterized by low estradiol, low progesterone: LH frequency, 7.0±1.8 pulses/12 h; LH amplitude, 2.3±1.0 IU/L [mean±2 SD]) (37, 38). In Subjects 2, 4, and 5, one pulse was observed in the sampling interval (7-8 hours; mean LH amplitude 1.5±0.8 mIU/mL) (FIG. 7B). In Subject 3, no pulses were observed during the study. In addition, the LH levels of Subjects 3, 4, and 5 demonstrated slow decay at the beginning of the sampling interval, suggesting that an LH secretory event had occurred before the start of the study. Thus, all subjects demonstrated an abnormally low frequency of LH secretory events. Upon repeat testing in 2016, study subjects (Subjects 3, 4, 5) again were amenorrheic with low but detectable estradiol levels off hormonal medications. All studies recapitulated the same endogenous LH patterns observed in 2010, with low-frequency LH secretory events and a mean LH amplitude of 1.3±1.1 mIU/mL (FIG. 8B).

In contrast, Subject 1, the healthy sister with a heterozygous protein truncating variant in TAC3, underwent blood sampling on Day 4 of the menstrual cycle (early follicular phase; EFP). She exhibited 11 LH pulses in 12 hours with a mean LH pulse amplitude of 0.46±0.25 mIU/mL (FIG. 7C) (healthy early follicular phase women: frequency, 7.0±1.8 pulses/12 h; amplitude, 2.3±1.0 IU/L [mean±2 SD]) (19, 20).

Kisspeptin Boluses: IHH Individuals without NKB Respond to Kisspeptin

All subjects responded to kisspeptin with an LH pulse (FIG. 8B). Two study subjects received three kisspeptin boluses and demonstrated an LH pulse following kisspeptin in 5 of the 6 boluses. The one exception occurred when kisspeptin was administered immediately following an endogenous LH peak resulting in a prolonged single peak (FIG. 8B, Subject 5). Consistent with this responsiveness, all subjects demonstrated adequate pituitary priming, indicating no pituitary defect that could impair kisspeptin responsiveness (LH pulse amplitude following GnRH administration: Subject 3: 1.6 mIU/mL, Subject 4: 5.1 mIU/mL, Subject 5: 3.0 mIU/mL).

Kisspeptin Infusion: No Pulsatile LH Secretion

Subject 3 received a kp-10 infusion (9.5 nmol/kg/hr) for 12 hours and no LH pulses were detected. There was a modest increase in mean LH during the infusion (baseline: 0.46±0.24 mIU/mL; kp-10 infusion: 0.63±0.08 mIU/mL; p<0.0001) (FIGS. 8B & 9B). Mean FSH levels also increased as compared to baseline (baseline: 1.9±0.2 mIU/mL; kp-10 infusion: 2.4±0.1 mIU/mL; p<0.001). After the kp-10 infusion, Subject 3 received an IVB of GnRH resulting in an LH pulse of comparable amplitude to that observed in baseline study the prior day (baseline, 1.6 mIU/mL; after kp-10 infusion, 2.5 mIU/mL).

Naloxone Infusion: Blocking Dynorphin with Naloxone Increases LH & FSH Secretion and LH Pulse Frequency, but does not Amplify Kisspeptin-Induced LH Pulses

Subjects 4 and 5 received the non-selective opioid antagonist, NLX, as well as escalating boluses of kisspeptin (0.24, 0.72, 2.4 nmol/kg) to determine the effect of blocking dynorphin signaling on endogenous and kisspeptin-stimulated LH secretory patterns. Both studies demonstrated increased mean LH levels during NLX infusion as compared to baseline (Subject 4—baseline: 1.44±0.76 mIU/mL, NLX: 2.82±0.54 mIU/mL, p<0.00001; Subject 5—baseline: 0.6±0.25 mIU/mL, NLX: 1.1±0.37 mIU/mL, p<0.00001, across matched time points) (FIG. 8B, 10B). For the study subject in which a complete LH sampling on and off NLX infusion allowed comparison, Subject 4, LH pulse frequency increased from one pulse in 6 hours (FIG. 8B) to four pulses in 6 hours (FIG. 10B). Mean FSH levels also increased as compared to baseline (Subject 4—baseline: 3.7±0.3 mIU/mL, NLX: 5.0±0.9 mIU/mL; p<0.01; Subject 5—baseline: 3.3±0.3 mIU/mL, NLX: 5.1±0.1 mIU/mL; p<0.0001). There was no consistent change in LH pulse amplitude (Subject 4—baseline: 2.59 mIU/, NLX: 0.45±0.29 mIU/mL; Subject 5—baseline: 0.82 mIU/mL, NLX: 1.22 and 1.39 mIU/mL). NLX infusions, which block dynorphin by inhibiting opioid tone, increase gonadotropin secretion and improve LH pulse frequency in individuals with IHH due to loss of NKB signaling.

Subjects 4 and 5 also received escalating boluses of kp-10 (0.24, 0.72, 2.4 nmol/kg) which were followed by an LH pulse, recapitulating results seen off NLX (FIG. 8B, 10B). There was no significant difference in the change in kisspeptin-induced LH response on or off NLX and there was no clear dose-response relationship; although the small number of boluses at each dose limited the ability to assess such a relationship.

Kisspeptin Boluses Stimulate LH Release in Peripubertal and Adult WT and NKB-Deficient (Tac2 KO) Mice

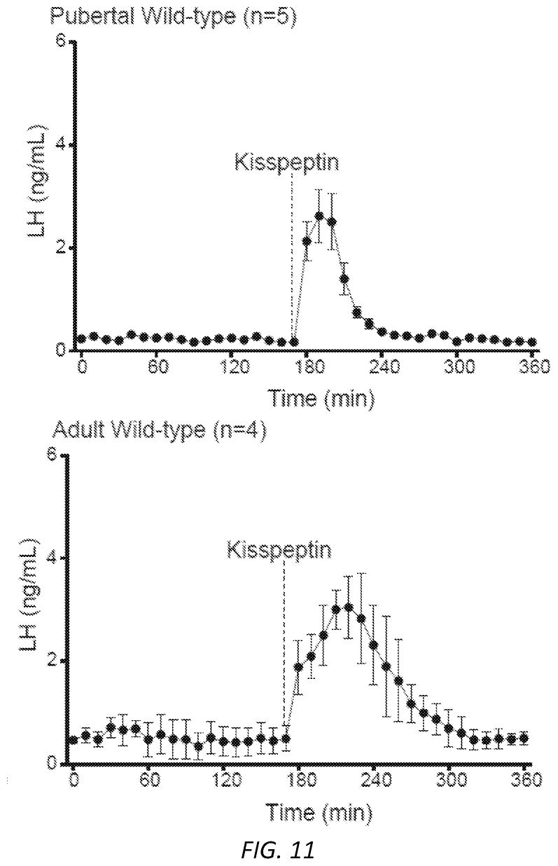

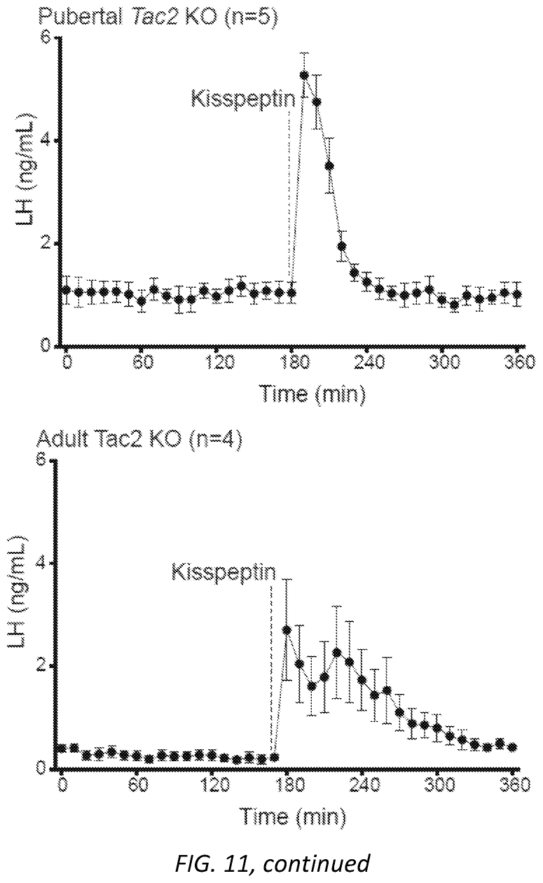

To corroborate the findings in IHH patients, we conducted experiments in Tac2 KO and WT control female mice. Peripheral administration of kp-10 elicited a robust increase in LH in all animal groups regardless of age and genotype. Interestingly, peripubertal Tac2 KO female mice, lacking NKB, displayed a higher magnitude of LH release (5.29±0.43 ng/ml, n=5) than control females (2.67±0.48 ng/ml, n=5; p<0.01) (FIG. 11). However, LH returned to baseline faster in Tac2 KO mice (52±3.72 min after injection, n=5) than in WT control (68±3.72 min, n=5; p<0.01). Adult WT mice displayed the expected LH pulse in response to kp-10, while the Tac2 KO mice that responded to kp-10 showed a bi-phasic response, displaying two overlapping peaks of LH (FIG. 10). In both adult groups, the induction of LH release appeared more sustained than in peripubertal mice (peripubertal WT: 68±3.742 min, n=5 vs adult WT142.5±4.78 min after injection, n=4, p<0.0001; peripubertal Tac2 KO: 52±3.742, n=5 vs adult Tac2 KO 156.7±3.33 min, n=3, p=0.07).

Naloxone Increases Pulsatile LH Release in Adult OVX WT and Tac2 KO Mice

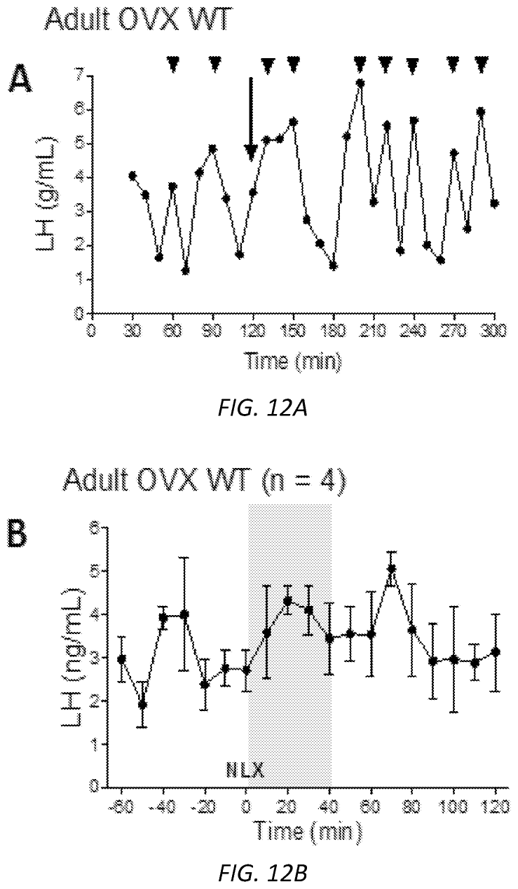

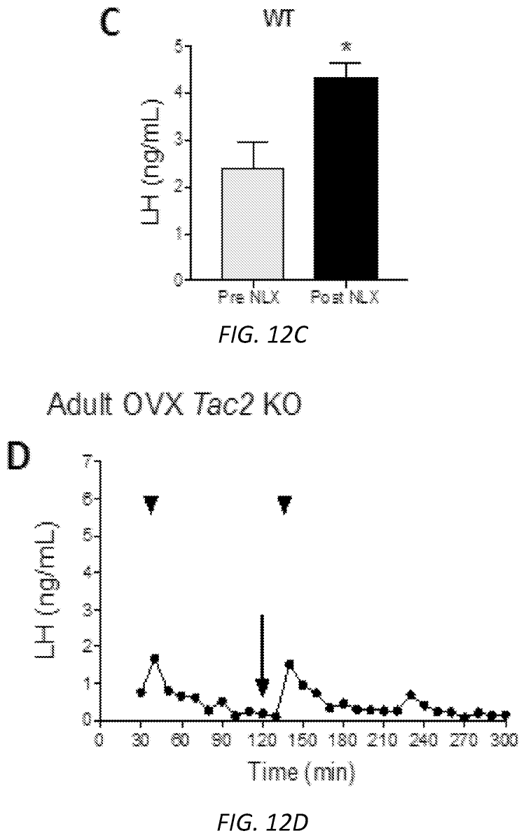

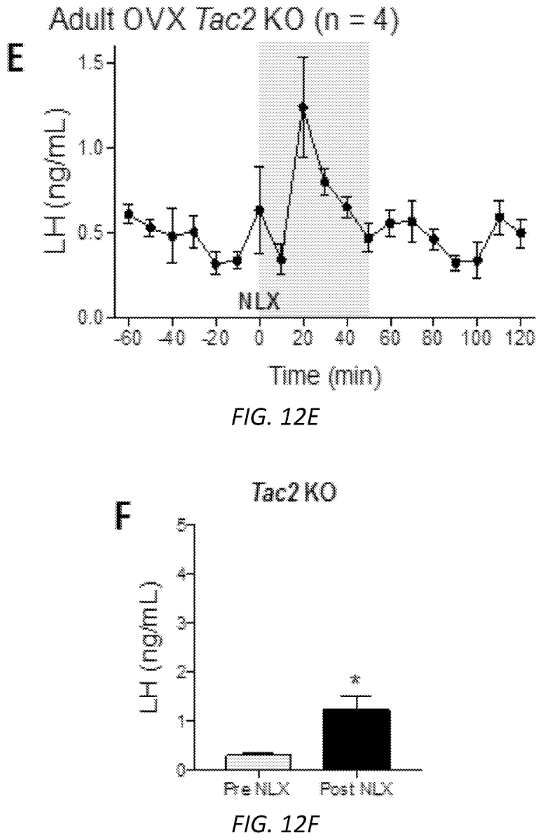

To determine the role of the opiatergic (dynorphin) influence on kisspeptin signaling in the absence of NKB, we examined the effects of NLX, which blocks dynorphin, on LH secretion. Peripheral administration of NLX 5 mg/kg induced an increase in LH in both WT (FIG. 12A-C) and Tac2 KO female mice (FIG. 12D-E) within 20 min of administration (WT: 20 min pre-NLX, 2.37±0.59, n=4 vs 20 min post NLX, 4.31±0.32, n=4; p<0.05. Tac2 KO: 20 min pre-NLX, 0.31±0.06, n=4 vs 20 min post NLX, 1.22±0.29, n=4, p<0.05).

After NLX administration, WT mice responded with an increase in the duration of the following LH pulse post-NLX administration (pre-NLX: WT 25±2.67 min, n=3; Tac2 KO 23.33±2.10 min, n=3, p=0.13; post-NLX: WT: 83.33±12.02 min, n=3; Tac2 KO: 30±5.77 min, n=3; p<0.01) (FIG. 12A). In addition, the increase in duration in the post-NLX LH pulse was accompanied by a pronounced and longer inter-pulse interval in WT mice (WT inter-pulse interval pre-NLX 25.38±1.83 min; WT inter-pulse interval post-NLX 46.67±3.33 min, p<0.0002).

Tac2 KO animals displayed a markedly reduced LH baseline and number of pulses than in OVX controls (0-1 LH pulses in 120 min pre-NLX). The administration of NLX induced a robust LH pulse that occurred 20 min after treatment in all cases, with a peak that reached a two-fold increase compared to baseline (pre-NLX: 0.31±0.06 mIU/mL; post-NLX: 1.2±0.28 mIU/mL, p<0.02). While the limited number of LH pulses precluded an analysis of inter-pulse intervals; data suggest that NLX did not increase the duration of the LH pulse (pre-NLX Tac2 KO 23.33±2.10 min, n=3, post-NLX: Tac2 KO: 30±5.77 min, n=3, p>0.05) (FIG. 12D-E).

### REFERENCES

1. Palmert M R, Dunkel L. Clinical practice. Delayed puberty. N Engl J Med. 2012; 366(5):443-453.
2. Wei C, Crowne E C. Recent advances in the understanding and management of delayed puberty. Arch Dis Child. 2015.
3. Sedlmeyer I L, Palmert M R. Delayed puberty: analysis of a large case series from an academic center. J Clin Endocrinol Metab. 2002; 87(4):1613-1620.
4. Lawaetz J G, Hagen C P, Mieritz M G, Blomberg Jensen M, Petersen J H, Juul A. Evaluation of 451 Danish boys with delayed puberty: diagnostic use of a new puberty nomogram and effects of oral testosterone therapy. J Clin Endocrinol Metab. 2015; 100(4):1376-1385.
5. Varimo T, Miettinen P J, Kansakoski J, Raivio T, Hero M. Congenital hypogonadotropic hypogonadism, functional hypogonadotropism or constitutional delay of growth and puberty? An analysis of a large patient series from a single tertiary center. Hum Reprod. 2017; 32(1):147-153.
6. Zhu J, Chan Y M. Adult Consequences of Self-Limited Delayed Puberty. Pediatrics. 2017; 139(6).
7. Balasubramanian R, Crowley W F, Jr. Isolated Gonadotropin-Releasing Hormone (GnRH) Deficiency. In: Adam M P, Ardinger H H, Pagon R A, Wallace S E, Bean L J H, Stephens K, Amemiya A, eds. GeneReviews(®). Seattle (Wash.)1993.
8. Harrington J, Palmert M R. Clinical review: Distinguishing constitutional delay of growth and puberty from isolated hypogonadotropic hypogonadism: critical appraisal of available diagnostic tests. J Clin Endocrinol Metab 2012; 97(9):3056-3067.
9. Adan L, Lechevalier P, Couto-Silva A C, Boissan M, Trivin C, Brailly-Tabard S, Brauner R. Plasma inhibin B and antimullerian hormone concentrations in boys: discriminating between congenital hypogonadotropic hypogonadism and constitutional pubertal delay. Med Sci Monit. 2010; 16(11): CR511-517.
10. Binder G, Schweizer R, Blumenstock G, Braun R. Inhibin B plus L H vs GnRH agonist test for distinguishing constitutional delay of growth and puberty from isolated hypogonadotropic hypogonadism in boys. Clin Endocrinol (Oxf) 2015; 82(1):100-105.
11. Rohayem J, Nieschlag E, Kliesch S, Zitzmann M. Inhibin B, A M H, but not INSL3, IGF1 or DHEAS support differentiation between constitutional delay of growth and puberty and hypogonadotropic hypogonadism. Andrology. 2015; 3(5):882-887.
12. Harter C J L, Kavanagh G S, Smith J T. The role of kisspeptin neurons in reproduction and metabolism. J Endocrinol. 2018; 238(3):R173-R183.
13. Dhillo W S, Chaudhri O B, Patterson M, Thompson E L, Murphy K G, Badman M K, McGowan B M, Amber V, Patel S, Ghatei M A, Bloom S R. Kisspeptin-54 stimulates the hypothalamic-pituitary gonadal axis in human males. J Clin Endocrinol Metab. 2005; 90(12):6609-6615.
14. Dhillo W S, Chaudhri O B, Thompson E L, Murphy K G, Patterson M, Ramachandran R, Nijher G K, Amber V, Kokkinos A, Donaldson M, Ghatei M A, Bloom S R. Kisspeptin-54 stimulates gonadotropin release most potently during the preovulatory phase of the menstrual cycle in women. J Clin Endocrinol Metab 2007; 92(10):3958-3966.
15. Chan Y M, Butler J P, Pinnell N E, Pralong F P, Crowley W F, Jr., Ren C, Chan K K, Seminara S B. Kisspeptin resets the hypothalamic GnRH clock in men. J Clin Endocrinol Metab 2011; 96(6):E908-915.
16. George J T, Veldhuis J D, Roseweir A K, Newton C L, Faccenda E, Millar R P, Anderson R A. Kisspeptin-10 is a potent stimulator of L H and increases pulse frequency in men. J Clin Endocrinol Metab. 2011; 96(8):E1228-1236.
17. Jayasena C N, Nijher G M, Comninos A N, Abbara A, Januszewki A, Vaal M L, Sriskandarajah L, Murphy K G, Farzad Z, Ghatei M A, Bloom S R, Dhillo W S. The effects of kisspeptin-10 on reproductive hormone release show sexual dimorphism in humans. J Clin Endocrinol Metab. 2011; 96(12):E 1963-1972.
18. Chan Y M, Butler J P, Sidhoum V F, Pinnell N E, Seminara S B. Kisspeptin administration to women: a window into endogenous kisspeptin secretion and GnRH responsiveness across the menstrual cycle. J Clin Endocrinol Metab. 2012.
19. Chan Y M, Lippincott M F, Butler J P, Sidhoum V F, Li C X, Plummer L, Seminara S B. Exogenous kisspeptin administration as a probe of GnRH neuronal function in patients with idiopathic hypogonadotropic hypogonadism. J Clin Endocrinol Metab. 2014; 99(12):E2762-2771.
20. Chan Y M, Lippincott M F, Kusa T O, Seminara S B. Divergent responses to kisspeptin in children with delayed puberty. JCI Insight. 2018; 3(8).
21. —BLANK—
22. Richards S, Aziz N, Bale S, Bick D, Das S, Gastier-Foster J, Grody W W, Hegde M, Lyon E, Spector E, Voelkerding K, Rehm H L, Committee ALQA. Standards and guidelines for the interpretation of sequence variants: a joint consensus recommendation of the American College of Medical Genetics and Genomics and the Association for Molecular Pathology. Genet Med. 2015; 17(5):405-424.
23. Boyar R, Finkelstein J, Roffwarg H, Kapen S, Weitzman E, Hellman L. Synchronization of augmented luteinizing hormone secretion with sleep during puberty. N Engl J Med. 1972; 287(12):582-586.
24. Zhu J, Choa R E, Guo M H, Plummer L, Buck C, Palmert M R, Hirschhorn J N, Seminara S B, Chan Y M. A shared genetic basis for self-limited delayed puberty and idiopathic hypogonadotropic hypogonadism. J Clin Endocrinol Metab. 2015; 100(4):E646-654.
25. Young J, George J T, Tello J A, Francou B, Bouligand J, Guiochon-Mantel A, Brailly-Tabard S, Anderson R A, Millar R P. Kisspeptin restores pulsatile L H secretion in patients with neurokinin B signaling deficiencies: physiological, pathophysiological and therapeutic implications. Neuroendocrinology. 2013; 97(2):193-202.
26. Lippincott M F, Leon S, Chan Y M, Fergani C, Talbi R, Farooqi I S, Jones C M, Arlt W, Stewart S E, Cole T R, Terasawa E, Hall J E, Shaw N D, Navarro V M, Seminara S B. Hypothalamic Reproductive Endocrine Pulse Generator Activity Independent of Neurokinin B and Dynorphin Signaling. J Clin Endocrinol Metab. 2019.
27. Topaloglu A K, Reimann F, Guclu M, Yalin A S, Kotan L D, Porter K M, Serin A, Mungan N O, Cook J R, Ozbek M N, Imamoglu S, Akalin N S, Yuksel B, O'Rahilly S, Semple R K. TAC3 and TACR3 mutations in familial hypogonadotropic hypogonadism reveal a key role for Neurokinin B in the central control of reproduction. Nat Genet. 2009; 41(3):354-358.
28. Gianetti E, Tusset C, Noel S D, Au M G, Dwyer A A, Hughes V A, Abreu A P, Carroll J, Trarbach E, Silveira L F, Costa E M, de Mendonca B B, de Castro M, Lofrano A, Hall J E, Bolu E, Ozata M, Quinton R, Amory J K, Stewart S E, Arlt W, Cole T R, Crowley W F, Kaiser U B, Latronico A C, Seminara S B. TAC3/TACR3 mutations reveal preferential activation of gonadotropin-releasing hormone release by neurokinin B in neonatal life followed by reversal in adulthood. J Clin Endocrinol Metab. 2010; 95(6):2857-2867.
29. Tusset C, Noel S D, Trarbach E B, Silveira L F, Jorge A A, Brito V N, Cukier P, Seminara S B, Mendonca B B, Kaiser U B, Latronico A C. Mutational analysis of TAC3 and TACR3 genes in patients with idiopathic central pubertal disorders. Arq Bras Endocrinol Metabol. 2012; 56(9):646-652.
30. Topaloglu A K, Tello J A, Kotan L D, Ozbek M N, Yilmaz M B, Erdogan S, Gurbuz F, Temiz F, Millar R P, Yuksel B. Inactivating KISS1 mutation and hypogonadotropic hypogonadism. N Engl J Med. 2012; 366(7):629-635.
31. Schally A V, Arimura A, Kastin A J, Matsuo H, Baba Y, Redding T W, Nair R M, Debeljuk L, White W F 1971 Gonadotropin-releasing hormone: one polypeptide regulates secretion of luteinizing and follicle-stimulating hormones. Science 173:1036-1038
32. Spratt D I, Carr D B, Merriam G R, Scully R E, Rao P N, Crowley W F, Jr. 1987 The spectrum of abnormal patterns of gonadotropin-releasing hormone secretion in men with idiopathic hypogonadotropic hypogonadism: clinical and laboratory correlations. J Clin Endocrinol Metab 64:283-291
33. Shaw N D, Seminara S B, Welt C K, Au M G, Plummer L, Hughes V A, Dwyer A A, Martin K A, Quinton R, Mericq V, Merino P M, Gusella J F, Crowley W F, Jr., Pitteloud N, Hall J E 2011 Expanding the phenotype and genotype of female GnRH deficiency. J Clin Endocrinol Metab 96:E566-576
34. Moore A M, Coolen L M, Porter D T, Goodman R L, Lehman M N 2018 KNDy Cells Revisited. Endocrinology 159:3219-3234
35. Chan Y M, Broder-Fingert S, Paraschos S, Lapatto R, Au M, Hughes V, Bianco S D, Min L, Plummer L, Cerrato F, De Guillebon A, Wu I H, Wahab F, Dwyer A, Kirsch S, Quinton R, Cheetham T, Ozata M, Ten S, Chanoine J P, Pitteloud N, Martin K A, Schiffmann R, Van der Kamp H J, Nader S, Hall J E, Kaiser U B, Seminara S B 2011 GnRH-deficient phenotypes in humans and mice with heterozygous variants in KISS1/Kiss1. J Clin Endocrinol Metab 96:E1771-1781
36. Lapatto R, Pallais J C, Zhang D, Chan Y M, Mahan A, Cerrato F, Le W W, Hoffman G E, Seminara S B 2007 Kiss1−/− mice exhibit more variable hypogonadism than Gpr54−/− mice. Endocrinology 148:4927-4936
37. Seminara S B, Messager S, Chatzidaki E E, Thresher R R, Acierno J S, Jr., Shagoury J K, Bo-Abbas Y, Kuohung W, Schwinof K M, Hendrick A G, Zahn D, Dixon J, Kaiser U B, Slaugenhaupt S A, Gusella J F, O'Rahilly S, Carlton M B, Crowley W F, Jr., Aparicio S A, Colledge W H 2003 The GPR54 gene as a regulator of puberty. N Engl J Med 349:1614-1627
38. de Roux N, Genin E, Carel J C, Matsuda F, Chaussain J L, Milgrom E 2003 Hypogonadotropic hypogonadism due to loss of function of the KiSS1-derived peptide receptor GPR54. Proc Natl Acad Sci USA 100:10972-10976
39. Funes S, Hedrick J A, Vassileva G, Markowitz L, Abbondanzo S, Golovko A, Yang S, Monsma F J, Gustafson E L 2003 The KiSS-1 receptor GPR54 is essential for the development of the murine reproductive system. Biochem Biophys Res Commun 312:1357-1363
40. Topaloglu A K, Reimann F, Guclu M, Yalin A S, Kotan L D, Porter K M, Serin A, Mungan N O, Cook J R, Ozbek M N, Imamoglu S, Akalin N S, Yuksel B, O'Rahilly S, Semple R K 2009 TAC3 and TACR3 mutations in familial hypogonadotropic hypogonadism reveal a key role for Neurokinin B in the central control of reproduction. Nature genetics 41:354-358
41. Topaloglu A K, Tello J A, Kotan L D, Ozbek M N, Yilmaz M B, Erdogan S, Gurbuz F, Temiz F, Millar R P, Yuksel B 2012 Inactivating KISS1 mutation and hypogonadotropic hypogonadism. N Engl J Med 366:629-635
42. d'Anglemont de Tassigny X, Fagg L A, Dixon J P, Day K, Leitch H G, Hendrick A G, Zahn D, Franceschini I, Caraty A, Carlton M B, Aparicio S A, Colledge W H 2007 Hypogonadotropic hypogonadism in mice lacking a functional Kiss1 gene. Proc Natl Acad Sci USA 104:10714-10719
43. Goodman R L, Coolen L M, Anderson G M, Hardy S L, Valent M, Connors J M, Fitzgerald M E, Lehman M N 2004 Evidence that dynorphin plays a major role in mediating progesterone negative feedback on gonadotropin-releasing hormone neurons in sheep. Endocrinology 145:2959-2967
44. Foradori C D, Goodman R L, Adams V L, Valent M, Lehman M N 2005 Progesterone increases dynorphin a concentrations in cerebrospinal fluid and preprodynorphin messenger ribonucleic Acid levels in a subset of dynorphin neurons in the sheep. Endocrinology 146:1835-1842
45. Shoupe D, Mishell D R, Jr., Fossum G, Bopp B L, Spitz I M, Lobo R A 1990 Antiprogestin treatment decreases midluteal luteinizing hormone pulse amplitude and primarily exerts a pituitary inhibition. American journal of obstetrics and gynecology 163:1982-1985
46. Goodman R L, Lehman M N, Smith J T, Coolen L M, de Oliveira C V, Jafarzadehshirazi M R, Pereira A, Iqbal J, Caraty A, Ciofi P, Clarke I J 2007 Kisspeptin neurons in the arcuate nucleus of the ewe express both dynorphin A and neurokinin B. Endocrinology 148:5752-5760
47. Han S Y, McLennan T, Czieselsky K, Herbison A E 2015 Selective optogenetic activation of arcuate kisspeptin neurons generates pulsatile luteinizing hormone secretion. Proc Natl Acad Sci USA 112:13109-13114
48. Clarkson J, Han S Y, Piet R, McLennan T, Kane G M, Ng J, Porteous R W, Kim J S, Colledge W H, Iremonger K J, Herbison A E 2017 Definition of the hypothalamic GnRH pulse generator in mice. Proc Natl Acad Sci USA 114:E10216-E10223
49. Sidhoum V F, Chan Y M, Lippincott M F, Balasubramanian R, Quinton R, Plummer L, Dwyer A, Pitteloud N, Hayes F J, Hall J E, Martin K A, Boepple P A, Seminara S B 2014 Reversal and relapse of hypogonadotropic hypogonadism: resilience and fragility of the reproductive neuroendocrine system. J Clin Endocrinol Metab 99:861-870
50. Sykiotis G P, Plummer L, Hughes V A, Au M, Durrani S, Nayak-Young S, Dwyer A A, Quinton R, Hall J E, Gusella J F, Seminara S B, Crowley W F, Jr., Pitteloud N 2010 Oligogenic basis of isolated gonadotropin-releasing hormone deficiency. Proc Natl Acad Sci USA 107:15140-15144
51. Lek M, Karczewski K, Minikel E, Samocha K, Banks E, Fennell T, O'Donnell-Luria A, Ware J, Hill A, Cummings B, Tukiainen T, Birnbaum D, Kosmicki J, Duncan L, Estrada K, Zhao F, Zou J, Pierce-Hoffman E, Cooper D, DePristo M, Do R, Flannick J, Fromer M, Gauthier L, Goldstein J, Gupta N, Howrigan D, Kiezun A, Kurki M, Levy Moonshine A, Natarajan P, Orozco L, Peloso G, Poplin R, Rivas M, Ruano-Rubio V, Ruderfer D, Shakir K, Stenson P, Stevens C, Thomas B, Tiao G, Tusie-Luna M, Weisburd B, Won H-H, Yu D, Altshuler D, Ardissino D, Boehnke M, Danesh J, Roberto E, Florez J, Gabriel S, Getz G, Hultman C, Kathiresan S, Laakso M, McCarroll S, McCarthy M, McGovern D, McPherson R, Neale B, Palotie A, Purcell S, Saleheen D, Scharf J, Sklar P, Patrick S, Tuomilehto J, Watkins H, Wilson J, Daly M, MacArthur D 2015 Analysis of protein-coding genetic variation in 60,706 humans. bioRxiv
52. Adzhubei I A, Schmidt S, Peshkin L, Ramensky V E, Gerasimova A, Bork P, Kondrashov A S, Sunyaev S R 2010 A method and server for predicting damaging missense mutations. Nature methods 7:248-249
53. Ng P C, Henikoff S 2003 SIFT: Predicting amino acid changes that affect protein function. Nucleic acids research 31:3812-3814
54. Schwarz J M, Rodelsperger C, Schuelke M, Seelow D 2010 MutationTaster evaluates disease-causing potential of sequence alterations. Nature methods 7:575-576
55. Thomas P D, Kejariwal A, Guo N, Mi H, Campbell M J, Muruganujan A, Lazareva-Ulitsky B 2006 Applications for protein sequence-function evolution data: mRNA/protein expression analysis and coding SNP scoring tools. Nucleic acids research 34:W645-650
56. Doty R L, Marcus A, Lee W W 1996 Development of the 12-item Cross-Cultural Smell Identification Test (CC-SIT). The Laryngoscope 106:353-356
57. Doty R L, Shaman P, Dann M 1984 Development of the University of Pennsylvania Smell Identification Test: a standardized microencapsulated test of olfactory function. Physiology & behavior 32:489-502
58. Chan Y M, Lippincott M F, Butler J P, Sidhoum V F, Li C X, Plummer L, Seminara S B 2014 Exogenous kisspeptin administration as a probe of GnRH neuronal function in patients with idiopathic hypogonadotropic hypogonadism. J Clin Endocrinol Metab 99:E2762-2771
59. Young J, George J T, Tello J A, Francou B, Bouligand J, Guiochon-Mantel A, Brailly-Tabard S, Anderson R A, Millar R P 2012 Kisspeptin Restores Pulsatile L H Secretion in Patients with Neurokinin B Signaling Deficiencies: Physiological, Pathophysiological and Therapeutic Implications. Neuroendocrinology
60. Dwyer A A, Hayes F J, Plummer L, Pitteloud N, and Crowley W F, Jr. The Long-Term Clinical Follow-Up and Natural History of Men with Adult-Onset Idiopathic Hypogonadotropic Hypogonadism. J Clin Endocrinol Metab. 2010 September; 95(9): 4235-4243.
61. Lee J H, Miele M E, Hicks D J, Phillips K K, Trent J M, Weissman B E & Welch D R 1996 KiSS-1, a novel human malignant melanoma metastasis-suppressor gene. Journal of the National Cancer Institute 88 1731-1737.
62. Ohtaki T, Shintani Y, Honda S, Matsumoto H, Hori A, Kanehashi K, Terao Y, Kumano S, Takatsu Y, Masuda Y, et al. 2001 Metastasis suppressor gene KiSS-1 encodes peptide ligand of a G-protein-coupled receptor. Nature 411 613-617.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject who has a reproductive endocrine dysfunction, optionally pathologic hypogonadotropic hypogonadotropism, the method comprising administering a therapeutically effective amount of (i) a plurality of doses of kisspeptin or a kisspeptin analog, and/or (ii) an opioid antagonist or mixed agonist-antagonist to the subject.

2. The method of claim 1, wherein the plurality of doses are administered at 1-6 hour intervals, preferably at 2 hour intervals, over at least 2-3 days and preferably over at least 1-12 months.

3. The method of claim 1, wherein each of the plurality of doses comprises a dose equivalent to 0.08-2.4 nmol/kg kisspeptin-10, administered via intravenous bolus (IVB).

4. The method of claim 3, wherein each of the plurality of doses comprises a dose equivalent to 0.2-0.3 nmol/kg kisspeptin-10, preferably 0.24 nmol/kg kisspeptin-10.

5. The method of claim 1, wherein the plurality of doses comprises at least one supraphysiologic dose equivalent to 2.4-24 nmol/kg kisspeptin-10.

6. The method of claim 5, wherein the at least one supraphysiologic dose is administered as a first dose, or first two or more doses, optionally all, of the plurality of doses.

7. The method of claim 1, further comprising administering a therapeutically effective amount of an opioid antagonist or mixed agonist-antagonist to the subject.

8. The method of claim 7, wherein the opioid antagonist is naloxone or naltrexone or the mixed agonist-antagonist is buprenorphine.

9. A method of identifying a subject as having pathologic hypogonadotropic hypogonadotropism, being at risk for pathologic hypogonadotropic hypogonadism, or having an attenuated form of pathologic hypogonadotropic hypogonadism, the method comprising:
measuring a baseline level of LH in the subject;
administering a stimulating dose of kisspeptin or a kisspeptin analog to the subject, optionally a dose comprising to 0.08-15 nmol/kg kisspeptin-10, administered via intravenous bolus (IVB) or a dose comprising 0.8-500 nmol/kg kisspeptin-10, administered via subcutaneous (SC) injection;
measuring at least one level of LH after administration of the stimulating dose;
comparing the baseline level of LH in the subject to the level of LH after administration of the stimulating dose, and
identifying a subject with delayed puberty who has a level of LH after administration of the stimulating dose that is not significantly different from the baseline level of LH as having pathologic hypogonadotropic hypogonadotropism.

10. The method of claim 9, further comprising administering a treatment for pathologic hypogonadotropic hypogonadotropism to the identified subject.

11. The method of claim 10, wherein the treatment comprises administering a plurality of doses of kisspeptin or a kisspeptin analog.

12. The method of claim 11, wherein the plurality of doses are administered at 1-6 hour intervals, preferably at 2 hour intervals, over at least 2-3 days and preferably over at least 1-12 months.

13. The method of claim 11, wherein each of the plurality of doses comprises a dose equivalent to 0.08-2.4 nmol/kg kisspeptin-10, administered via intravenous bolus (IVB).

14. The method of claim 13, wherein each of the plurality of doses comprises a dose equivalent to 0.2-0.3 nmol/kg kisspeptin-10, preferably 0.24 nmol/kg kisspeptin-10.

15. The method of claim 11, wherein the plurality of doses comprises at least one supraphysiologic dose equivalent to 2.4-24 nmol/kg kisspeptin-10.

16. The method of claim 15, wherein the at least one supraphysiologic dose is administered as a first dose, or first two or more doses, optionally all, of the plurality of doses.

17. The method of claim 10, comprising administering a therapeutically effective amount of an opioid antagonist or mixed agonist-antagonist to the subject.

18. The method of claim 17, wherein the opioid antagonist is naloxone or naltrexone or the mixed agonist-antagonist is buprenorphine.

19. The method of claim 1, wherein the treatment further comprises administering gonadal steroid replacement therapy.

20. The method of claim 19, wherein the gonadal steroid replacement therapy comprises administering testosterone in males and estrogen and/or progesterone/progestin in females.

21. The method of claim 1, further comprising administration of one or more gonadotropins.

22.-32. (canceled)
